SEQUENCE LISTING (34 sequences)

SEQ ID NO: 1 - DNA, 11 bp, Artificial Sequence
  Control Strand
gaaggaggaga

SEQ ID NO: 2 - DNA, 11 bp, Artificial Sequence
  Linked to other strands to form hairpins
cttcctcctct

SEQ ID NO: 3 - DNA, 11 bp, Artificial Sequence
  8-aminoadenine component of hairpin strand
gaaggnggnga

SEQ ID NO: 4 - DNA, 11 bp, Artificial Sequence
  8-aminoguanine component of hairpin strand
gaagnagnaga

SEQ ID NO: 5 - DNA, 11 bp, Artificial Sequence
  8-aminohypoxyanthine component of hairpin strand
gaagnagnaga

SEQ ID NO: 6 - DNA, 11 bp, Artificial Sequence
  test sequence
aaaaaaaaaaa

SEQ ID NO: 7 - DNA, 11 bp, Artificial Sequence
  test sequence
ttttttttttt

SEQ ID NO: 8 - DNA, 11 bp, Artificial Sequence
  test strand
ccccctttttt

SEQ ID NO: 9 - DNA, 7 bp, Artificial Sequence
  test strand
tcctcct

SEQ ID NO: 10 - DNA, 10 bp, Artificial Sequence
  Strand for Stabilization testing
gaagnaggag

SEQ ID NO: 11 - DNA, 26 bp, Artificial Sequence
  test sequence
gaaggaggagatttttctcctccttc

SEQ ID NO: 12 - DNA, 26 bp, Artificial Sequence
  test sequence
gaagnagnanatttttctcctccttc

SEQ ID NO: 13 - DNA, 26 bp, Artificial Sequence
  test sequence
ganggnggngatttttctcctccttc

SEQ ID NO: 14 - DNA, 11 bp, Artificial Sequence
  test sequence
agaggaggaag

SEQ ID NO: 15 - DNA, 11 bp, Artificial Sequence
  test sequence
tgtggtggttg

SEQ ID NO: 16 - DNA, 11 bp, Artificial Sequence
  target sequence
tctcctccttc

SEQ ID NO: 17 - DNA, 10 bp, Artificial Sequence
  test sequence for triplex formation
cccttttccc

SEQ ID NO: 18 - DNA, 10 bp, Artificial Sequence
  test sequence
gggaaaaggg

SEQ ID NO: 19 - DNA, 10 bp, Artificial Sequence
  test sequence
gnnaaaanng

SEQ ID NO: 20 - DNA, 31 bp, Artificial Sequence
  polypyrimidine target
cgagtcattgtctcctccttcagtcatcgag

SEQ ID NO: 21 - DNA, 11 bp, Artificial Sequence
  test strand
gaagcnggnga

SEQ ID NO: 22 - DNA, 11 bp, Artificial Sequence
  test strand
cttcttcctct

SEQ ID NO: 23 - DNA, 11 bp, Artificial Sequence
  test sequence
cttcgtcctct

SEQ ID NO: 24 - DNA, 11 bp, Artificial Sequence
  test sequence
cttcatcctct

SEQ ID NO: 25 - DNA, 11 bp, Artificial Sequence
  test sequence
gaagtnggnga

SEQ ID NO: 26 - DNA, 16 bp, Artificial Sequence
  test sequence
tttttcttcctcctct

SEQ ID NO: 27 - DNA, 15 bp, Artificial Sequence
  test sequence
ttttgaaggnggnga

SEQ ID NO: 28 - DNA, 16 bp, Artificial Sequence
  test sequence
ggagggaaggnggnga

SEQ ID NO: 29 - DNA, 16 bp, Artificial Sequence
  test sequence
gtttcgaaggnggnga

SEQ ID NO: 30 - DNA, 11 bp, Artificial Sequence
  test sequence
tctcctgcttc

SEQ ID NO: 31 - DNA, 11 bp, Artificial Sequence
  test sequence
tctcctacttc

SEQ ID NO: 32 - DNA, 31 bp, Artificial Sequence; identical to SEQ ID NO: 20
  test sequence

SEQ ID NO: 33 - DNA, 31 bp, Artificial Sequence
  test sequence
ctcgatgactgaaggaggagacaatgactcg

SEQ ID NO: 34 - DNA, 11 bp, Artificial Sequence
  Strand containing 8-aminoadenine and 8-aminoguanine
gaagnngnnga

(19) United States
(12) Patent Application Publication
(10) Pub. No.: US 2004/0029160 A1
(43) Pub. Date: Feb. 12, 2004

(54) Parallel stranded duplexes of deoxyribonucleic acid and methods of use

(76) Inventors: Ramon Eritja, Barcelona (ES); Ramon G. Garcia, Heidelberg (DE)

Correspondence Address: CRAIG G. COCHENOUR, ESQ., BUCHANAN INGERSOLL, P.C., ONE OXFORD CENTRE, 20th FLOOR, 301 GRANT STREET, PITTSBURGH, PA, 15219, US

(21) Appl. No.: 10/446,201

(22) Filed: May 23, 2003

Related U.S. Application Data
(60) Provisional application No. 60/383,292, filed on May 24, 2002.

(51) Int. Cl. C12Q 1/68; C07H 21/04

(52) U.S. Cl. 435/6; 536/23.1

(57) ABSTRACT

A triplex comprising a hairpin having at least one polypyrimidine sequence linked to a complementary polypurine wherein the polypurine sequence is at least one 8-aminopurine such as 8-aminoadenine, 8-aminoguanine and 8-aminohypoxanthine, and at least one polypyrimidine target sequence that is complementary and antiparallel to the polypurine sequence. The polypurine sequence binds the polypyrimidine target sequence by forming a triplex helix. Methods for preparing the hairpins and for stabilizing the triplex are provided. Methods for targeting single-stranded oligonucleotides and DNA is described using the hairpins and triplexes of this invention.

### BENEFIT OF PRIOR PROVISIONAL APPLICATION

This utility patent application claims the benefit of priority of co-pending U.S. Provisional Patent Application Serial No. 60/383,292, filed May 24, 2002, entitled “Parallel Stranded Duplexes Of Deoxyribonucleic Acid And Methods Of Use” having the same named applicants as inventors, namely, Ramon Eritja and Ramon G. Garcia. The entire contents of U.S. Provisional Patent Application Serial No. 60/383,292 is incorporated by reference into this utility patent application.

### COMPUTER READABLE FORM AND SEQUENCE LISTING

Applicants state that the content of the sequence listing information recorded in computer readable form (CRF) as filed with this utility patent application is identical to the written paper sequence listing as filed with this utility patent application and contains no new matter as required by 37 CFR 1.821 (e-g) and 1.825 (b) and (d).

### BACKGROUND OF THE INVENTION

1. Field of the Invention

This invention relates to a novel triplex comprising a polypyrimidine sequence, a linker, and a polypurine sequence that is complementary to and parallel to the polypyrimidine sequence, and wherein the polypurine sequence comprises at least one 8-aminopurine, and a polypyrimidine target sequence that is complementary to and antiparallel to the polypurine sequence. Methods for preparing and using the triplex are also provided.

2. Description of the Background Art

DNA can form a large range of helical structures including duplexes, triplexes, and tetraplexes. The right-handed B-type duplex is the most common structure of DNA, but even now, decades after the discovery of the B-DNA, new double helical conformations of DNA are being described. Thus, those skilled in the art appreciate that DNA has great flexibility and exhibits a large polymorphism depending on sequence, chemical modifications, or alterations in the DNA environment.

Most DNA duplexes, including the well-known B and A forms, are antiparallel (i.e., one strand runs 5′→3′ and the other 3′→5′), but parallel arrangements have been found in both hairpins and linear DNAs. Sequences with propensity to form parallel DNAs have been found in specific chromosome regions, and could have an evolutionary role. Moreover, certain types of parallel-stranded DNA can be excellent templates for the formation of triplexes. This is very useful for biotechnological purposes, including antigene (targeting of genetic DNA by an artificial oligonucleotide) and antisense (targeting of natural messenger RNA by an artificial oligonucleotide) therapies.

Parallel DNA duplexes were first found in the crystal structure of a very short, mismatched DNA sequence intercalated by proflavine. Low resolution data of parallel-stranded duplex were found for longer pieces of RNA of sequence poly [d(A·U)], where the 2-position of adenines was modified by addition of bulky groups. The first structural model of polymeric parallel-stranded duplex DNA was derived by Pattabiraman, who on the basis of theoretical calculations designed a model for the parallel pairing of poly[d(A·T)] duplexes based on the reverse Watson-Crick motif. This model has been confirmed by low and high resolution experimental techniques on d(A·T) rich sequences.

The parallel-stranded duplex model early described by Pattabiraman and further refined by NMR data shows a general structure not far from the canonical antiparallel B-type helix. The bases are mostly perpendicular to the helix axis, there are two equivalent grooves, sugar units present puckerings in the South region, and the AT pairings are reverse Watson-Crick (FIG. 1). This structure—the parallel reverse Watson-Crick (rWC) duplex—is the most stable conformation for parallel-stranded helices rich in d(A·T) pairs, as demonstrated by Jovin and others using a variety of thermodynamic and spectroscopic techniques. The rWC double helix is less stable than comparable antiparallel helices, but it can be found in hairpins and linear DNAs designed to hinder the formation of the antiparallel d(A·T) helix. The presence of a few d(G·C) steps in the rWC double helix might be tolerated, but it destabilizes the duplex.

An alternative structure for parallel-stranded duplexes based on the Hoogsteen (H) recognition mode is also possible (FIG. 1). This would lead to a double helix (not yet described from a structural point of view) which might act as a template for triplex formation. Parallel-stranded DNA duplexes based on the H pairing occur in duplexes where purines are modified at position 2, which prevents both Watson-Crick and reverse Watson-Crick pairings, or in duplexes rich in d(G·C) (or d(G·G)) pairs. These latter duplexes can exist at neutral pH, but they are especially stable at low pH owing to the need to protonate the Hoogsteen cytosine (FIG. 1). The stability of the duplex can be also enhanced by DNA-binding drugs such as benzopyridoindole derivatives. Finally, as shown by Lavelle and Fresco and others, H-based parallel duplexes can be more stable than the canonical B-type antiparallel duplex under certain conditions.

Oligonucleotides bind in a sequence-specific manner to homopurine-homopyrimidine sequences of duplex and single-stranded DNA and RNA to form triplexes. Nucleic acid triplexes have wide applications in diagnosis, gene analysis and therapy, namely the extraction and purification of specific nucleotide sequences, control of gene expression, mapping of genomic DNA, induction of mutations in genomic DNA, detection of mutations in homopurine DNA sequences, site-directed mutagenesis, triplex-mediated inhibition of viral DNA integration, non-enzymatic ligation of double-helical DNA and quantification of polymerase chain reactions.

One of the main drawbacks of these applications is the low stability of triple helices especially in neutral conditions, and when the homopurine-homopyrimidine tracks have interruptions. A large effort has been made to design modified oligonucleotides and thus enhance triple helix stability in homopolymers and triplexes with interruptions in the homopurine-homopyrimidine tracks. Successful modifications of the nucleobases include molecules such as 5-methylcytidine, 5-methyl-2,6(1H,3H)-pyrimidinedione, and 2′-O-methylpseudoisocytidine.

Triplexes are typically formed by adding a triplex-forming oligonucleotide (TFO) to a duplex DNA. However, an alternative approach is based on the use of parallel-stranded duplexes. Accordingly, purine residues are linked to a pyrimidine chain of inverted polarity by 3′-3′ or 5′-5′ internucleotide junctions. Such parallel-stranded DNA hairpins have been synthesized and bind single-stranded DNA and RNA-targets by triplex formation, similar to the foldback all-pyrimidine hairpins that are known by those skilled in the art.

It will be appreciated by those skilled in the art that the structure of parallel-stranded DNAs is quite flexible and can change from H to rWC motifs depending on sequence, pH, and the presence of drugs. Low pH and high content of d(G·C) pairs favor the H-based structure, while the rWC helix is favored in d(A·T) rich sequences and at neutral or basic pH.

### SUMMARY OF THE INVENTION

In this invention the structure of parallel-stranded duplexes in mixed d(A·T) and d(G·C) sequences using state-of-the-art theoretical calculations and spectroscopic techniques were analyzed. This invention provides 8-amino derivatives to stabilize parallel duplexes that can be then used as templates for the formation of triple helices of DNA or DNA-RNA-DNA, that have a large impact in biotechnological and pharmaceutical research.

In one embodiment of this invention, a triplex is provided comprising a hairpin comprising at least one first polypyrimidine sequence, at least one linker, and at least one polypurine sequence, wherein at least one of the polypurine sequence is complementary to and parallel to the first polypyrimidine sequence, and the polypurine sequence comprising at least one 8-aminopurine; and at least one polypyrimidine target sequence, wherein at least one of the polypyrimidine target sequence is complementary to and antiparallel to the polypurine sequence, wherein the polypyrimidine target sequence and the hairpin are bound to each other. In a preferred embodiment of this invention, the triplex includes wherein the polypyrimidine target sequence comprises at least one purine interruption. In another embodiment of this invention, the triplex includes wherein the polypurine sequence of the hairpin comprises at least one pyrimidine interruption. In yet another embodiment of this invention, the triplex includes wherein the first polypyrimidine sequence of the hairpin comprises at least one purine interruption or an abasic interruption or an abasic model compound interruption. The triplex, as described herein, includes the linker that is at least one of a hexaethylene glycol, a tetrathymine, CTTTG, or GGAGG.

In a preferred embodiment of this invention, the triplex includes wherein the 8-aminopurine comprises 8-aminopurine.

In another preferred embodiment of this invention, the triplex includes wherein the 8-aminopurine comprises 8-aminoadenine.

In another preferred embodiment of this invention, the triplex includes where the 8-aminopurine comprises 8-aminohypoxanthine.

Another embodiment of this invention provides a method for preparing a hairpin containing at least one 8-aminopurine comprising preparing a pyrimidine strand; binding a linker to the 3′ end of the pyrimidine strand; preparing a purine strand comprising at least one 8-aminopurine; and preparing the hairpin by binding the 3′ end of the purine strand to the linker.

In another embodiment of this invention, a method for preparing a hairpin containing at least one 8-aminopurine is provided comprising preparing a purine strand comprising at least one 8-aminopurine; binding a linker to the 5′ end of the purine strand; preparing a pyrimidine strand; and preparing the hairpin by binding the 5′ end of the pyrimidine strand to the linker.

Another embodiment of this invention includes a hairpin comprising at least one first polypyrimidine sequence, at least one linker, and at least one polypurine sequence, wherein at least one of the polypurine sequence comprises at least one 8-aminopurine and wherein the polypurine sequence is complementary to and parallel to the first polypyrimidine sequence.

The present invention also provides a method for stabilizing a triplex comprising obtaining a triplex comprising a hairpin, wherein the hairpin comprises at least a first polypyrimidine sequence, at least one linker, and at least one polypurine sequence, wherein the polypurine sequence comprises at least one 8-aminopurine, and contacting the triplex with a sodium chloride solution or a solution containing magnesium or derivatives thereof..

In another embodiment of this invention, a triplex is provided comprising a hairpin comprising at least one first polypyrimidine sequence, at least one linker, and at least one first polypurine sequence wherein the polypurine sequence is complementary to. and antiparallel to the first polypyrimidine sequence, and the first polypurine sequence comprising at least one 8-aminopurine, and a target sequence wherein the target sequence is arranged in Hoogsteen orientation with respect to the hairpin.

In another embodiment of this invention, an oligonucleotide duplex is provided comprising two complementary oligonucleotide strands arranged in an anti-parallel Hoogsteen configuration.

The present invention also provides a method for stabilizing Hoogsteen duplexes comprising procuring a Hoogsteen duplex comprising at least one purine and stabilizing the Hoogsteen duplex by substituting at least one 8-aminopurine for at least one of the purine.

In yet another embodiment of this invention. a method for targeting a single-stranded oligonucleotide is provided comprising selecting a region on a single-stranded oligonucleotide, the region having either a first polypurine sequence target or a first polypyrimidine sequence target. preparing a hairpin wherein the hairpin comprises a second polypyrimidine sequence and a second polypurine sequence, wherein the second polypurine sequence comprises at least one 8-aminopurine and is complementary to the second polypyrimidine sequence, and targeting the region on the single-stranded oligonucleotide by contacting the hairpin with the first polypurine sequence target or the first polypyrimidine sequence target. In a preferred embodiment, this method includes wherein the single-stranded oligonucleotide is selected from the group consisting of cDNA, mRNA, tRNA, and rRNA.

Another embodiment of the present invention provides a method for targeting DNA comprising selecting a region on DNA, the region having either a first polypurine sequence target or a first polypyrimidine sequence target, preparing a hairpin wherein the hairpin comprises a second polypyrimidine sequence and a second polypurine sequence, wherein the second polypurine sequence comprises at least one 8-aminopurine and is complementary to the second polypyrimidine sequence, and targeting the region on the DNA by contacting the hairpin with the first polypurine sequence target or the first polypyrimidine sequence target.

### BRIEF DESCRIPTION OF THE DRAWINGS

FIG. 1 is a schematic representation of the Watson-Crick, reverse Watson-Crick, and Hoogsteen A·T pairings.

FIG. 2 shows the thermodynamic cycle used to compute the stabilization of parallel-stranded duplexes induced by the introduction of 8-amino derivatives.

FIG. 3 shows the MD-averaged structures of the Hoogsteen duplexes obtained in the A and B trajectories. The conformation of the Hoogsteen duplex in a B-type triplex is displayed for comparison.

FIG. 4 shows the final structures obtained in the three trajectories of the reverse Watson-Crick duplex. The structure generated from the experimental NMR structure (reference f in Table 1) is displayed for comparison.

FIG. 5 sets forth a classical molecular interaction potentials (cMIP; top) and solvation maps (bottom) for the canonical antiparallel duplex (left) and Hoogsteen parallel-stranded duplex (right). cMIP contours correspond to interaction energy of −5 to 5 kcal/mol (O⁺ was used as a probe). Solvation contours correspond to a density of 2 g/mL. For parallel duplexes cMIP and solvation maps were determined averaging over the A and B trajectories simultaneously.

FIG. 6 is a representation of protonated and wobble Hoogsteen 8AG-C dimers.

FIG. 7 sets forth sequences of parallel-stranded hairpins carrying 8-aminopurines of this invention: A^{N}, 8-aminoadenine; G^{N}, 8-aminopurine; I^{N}, 8-aminohypoxanthine; and a (EG)₆ hexaethylene glycol linker. Two anti-parallel duplexes used as control are also displayed.

FIG. 8 shows the dependence of Tm with pH for R-22 (SEQ ID NO: 1, SEQ ID NO: 2).B-22 (SEQ ID NO: 1, SEQ ID NO: 2) and two antiparallel duplexes D1 (SEQ ID NO: 1, SEQ ID NO: 2) and D2 (SEQ ID NO:1, SEQ ID NO: 9).

FIG. 9 shows: (A) CD spectra of hairpins B-22 (SEQ ID NO:1, SEQ ID NO:2), B-22A(SEQ ID NO:3, SEQ ID NO:2), B-22G (SEQ ID NO:4, SEQ ID NO:2), B-AT (SEQ ID NO: 6, SEQ ID NO:7), and an antiparallel duplex formed by B-22A control (SEQ ID NO:3, SEQ ID NO:8) (B-22 hairpin where the sequence of the pyrimidine strand is random) and a suitable single-stranded oligonucleotide (S11 WC) (SEQ ID NO: 16), and (B) CD spectra of B22A control (SEQ ID NO:3, SEQ ID NO:8) alone and after addition of the antiparallel complementary pyrimidine strand (0.1 M sodium phosphate pH 6.0, 50 mM NaCl, 10 mM MgCl₂).

FIG. 10 shows the exchangeable proton region of the NMR spectra of: d(3′-AGA^{N}GGA^{N}GGAAG-5′-(EG)₆-5′-CTTCCTCCTCT-3′) at T=50° C.

FIG. 11 shows the base pairing scheme of G: 8aminoG:C and T:8-aminoA:T.

FIG. 12 shows gel-shift analysis performed with s₁₁-GA (SEQ ID NO: 14) and s₁₁-GT (SEQ ID NO: 15), h₂₆ (SEQ ID NO: 11), h₂₆-3AG (SEQ ID NO: 12) and h₂₆-3AA (SEQ ID NO: 13).

FIG. 13 shows gel-shift analysis performed with hairpin RE-2AG (SEQ ID NO: 4, SEQ ID NO: 14) and its polypyrimidine target WC-11 mer (SEQ ID NO: 16).

FIG. 14 shows a scheme of binding a polypyrimidine single-stranded nucleic acid with hairpins of the present invention. Lower part: base-pairing schemes of triads containing 8-aminopurines.

FIG. 15 shows sequences of parallel-stranded hairpins carrying 8-aminopurines of this invention: A^{N}: 8-aminoadenine; G^{N}: 8-aminopurine; I^{N}: 8-aminohypoxanthine; and (EG)₆ : hexaethylenglycol linker, and GTTTC, GGAGG and TTTT linkers. Also shown is a hairpin of this invention containing an abasic model compound.

FIG. 16 sets forth root mean square deviations (RMSd in A) between the trajectories of the parallel Hoogsteen (Ho) and antiparallel Watson-Crick (WC) duplexes and their respective MD-averaged structures (top), and between the same trajectories and the MD-averaged structures of both duplexes in the antiparallel triplex (bottom). Bases at both ends were removed for RMSd calculations.

FIG. 17 shows binding of SEQ ID NO: 1, SEQ ID NO: 2) R-22A (SEQ ID NO: 3, SEQ ID NO: 2) and R-22G (SEQ ID NO:4, SEQ ID NO: 2) to WC-11 mer (SEQ ID NO: 16) (citric-phosphate buffer pH 6 of 100 mM Na⁺ ionic strength). Radiolabelled DNA target (10 nmol) was incubated at room temperature with 2-200 equivalents of cold hairpins R-22 (SEQ ID NO: 1, SEQ ID NO: 2), R-22A (SEQ ID NO: 3, SEQ ID NO: 2)and R-22G (SEQ ID NO: 4, SEQ ID NO: 2)and the mixtures were analyzed by 15% native polyacrylamide gel electrophoresis at room temperature.

FIG. 18 shows binding of hairpin R-22G (SEQ ID NO: 4, SEQ ID NO: 2) to single-stranded target T₃₁ (SEQ ID NO: 32) at pH 5.0. Left Side: The ³²P-labelled oligonucleotide was the target T₃₁ (SEQ ID NO: 32) and increasing (2×, 20×, 200×) amounts of cold B-22G were added. Right side: The ³²P-labelled oligonucleotide was the hairpin R-22G (SEQ ID NO: 4, SEQ ID NO: 2)and increasing (2×, 20×, 200×) amounts of cold T₃₁ (SEQ ID NO: 32) were added. Incubation time I hr at room temperature.

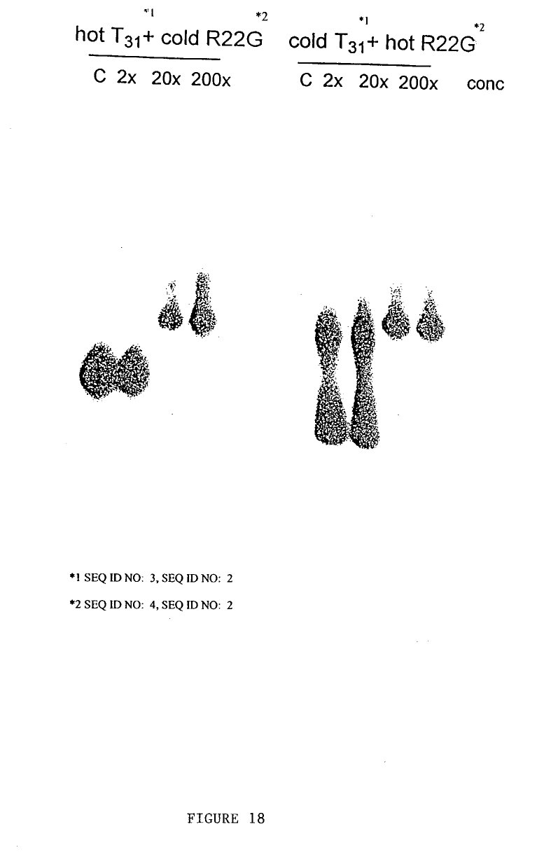

FIG. 19 shows the CD spectra of hairpins B-22 (SEQ ID NO: 1, SEQ ID NO: 2), B-22G (SEQ ID NO: 4, SEQ ID NO: 2) and B-22A(SEQ ID NO: 3, SEQ ID NO: 2) alone and together with their pyrimidine target WC-11 mer (SEQ ID NO: 16) (50 mM naCl, 10 mM MgCl₂, 0.1M sodium phosphate pH 6).

FIG. 20 shows the exchangeable proton region of the NMR spectra of triplex formed by B22A: d(3′-AGA^{N}GGA^{N}GGAAG-5′-(EG)₆-5′-CTTCCTCCTCT-3′) (SEQ ID NO: 3, SEQ ID NO: 2)and WC-11 mer (3′-CTTCCTCCTCT-5′) (SEQ ID NO: 16) at T=5° C.

FIG. 21 shows melting temperatures of triplexes formed by hairpins B-22A (SEQ ID NO:3, SEQ ID NO:2) and B-22G (SEQ ID NO: 4, SEQ ID NO:2) at various salt concentrations.

FIG. 22 shows the binding of hairpin R-22G (SEQ ID NO: 4, SEQ ID NO:2) to single and double-stranded targets by gel-shift experiments.

FIG. 23 shows the melting experiment on the triplex formed by B-22G (SEQ ID NO: 4, SEQ ID NO:2) and WC-11 mer (SEQ ID NO:16) followed by CD.

FIG. 24 shows Hoogsteen base pairs and parallel-stranded DNA.

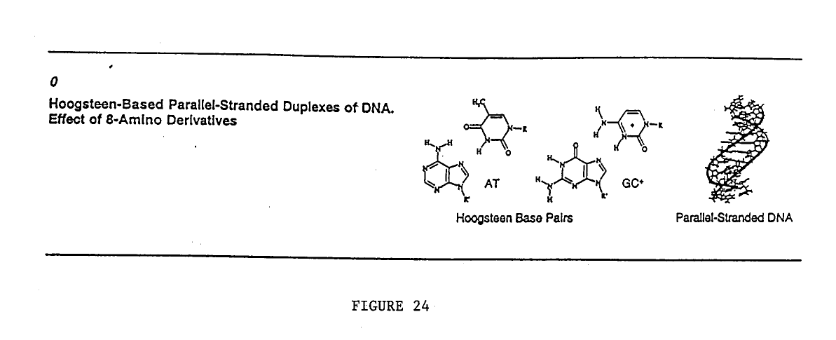

### DETAILED DESCRIPTION OF THE INVENTION

The present invention provides a triplex comprising a hairpin comprising at least one polypyrimidine sequence, at least one linker, and at least one polypurine sequence, wherein the polypurine sequence is complementary to and parallel to the polypyrimidine sequence, and wherein the polypurine sequence comprises at least one 8-aminopurine, and a polypyrimidine target sequence complementary to and antiparallel to the polypurine sequence. The polypyrimidine target sequence and the hairpin are bound to each other. The triplex includes wherein the polypyrimidine target sequence comprises at least one purine interruption. In another embodiment of this invention, the triplex includes wherein the polypurine sequence of the hairpin comprises at least one pyrimidine interruption. In another embodiment, the triplex as described herein includes wherein the first polypyrimidine sequence of the hairpin comprises at least one purine interruption or an abasic interruption or an abasic model compound interruption.

A method for preparing a hairpin containing at least one 8-aminopurine of this invention comprises preparing a pyrimidine strand, binding a linker to the 3′ end of the pyrimidine strand, preparing a purine strand comprising at least one 8-aminopurine, and preparing the hairpin by binding the 3′ end of the purine strand to the linker. In another embodiment the method for preparing a hairpin containing at least one 8-aminopurine comprises preparing a purine strand comprising at least one 8-aminopurine, binding a linker to the 5′ end of the purine strand, preparing a pyrimidine strand, and preparing the hairpin by binding the 5′ end of the pyrimidine strand to the linker.

This invention includes the hairpin as described herein comprising at least one first polypyrimidine sequence, at least one linker, and at least one polypurine sequence, wherein at least one of the polypurine sequence comprises at least one 8-aminopurine and wherein the polypurine sequence is complementary to and parallel to the first polypyrimidine sequence.

Another embodiment of this invention provides a triplex comprising a hairpin comprising at least one first polypyrimidine sequence, at least one linker, and at least one first polypurine sequence wherein the polypurine sequence is complementary to and antiparallel to the first polypyrimidine sequence, and the first polypurine sequence comprising at least one 8-aminopurine, and a target sequence wherein the target sequence is arranged in Hoogsteen orientation with respect to the hairpin. In a preferred embodiment of this invention the triplex includes wherein the target sequence comprises G and T bases or G and A bases.

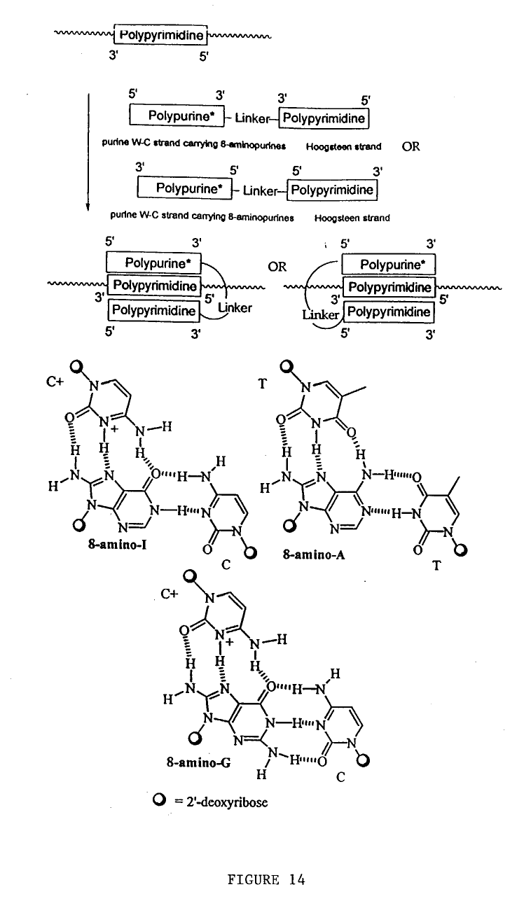

In a preferred embodiment of this invention, a triplex is provided comprising a hairpin comprising a polypyrimidine sequence, a linker, and a polypurine sequence, wherein the polypurine sequence is complementary to and parallel to the polypyrimidine sequence, wherein the polypurine sequence comprises at least one 8-aminopurine, and a polypyrimidine target sequence wherein the polypyrimidine target sequence is complementary to and antiparallel to the polypurine sequence. In the present invention, oligonucleotides containing 8-aminopurines replace natural purines in triplexes. The introduction of an amino group at position 8 of adenine and guanine increases the stability of the triple helix owing to the combined effect of the gain in one Hoogsteen purine-pyrimidine H-bond (FIG. 14) and to the ability of the amino group to be integrated into the “spine of hydration” located in the minor-Major groove of the triplex structure. The preparation and binding properties of oligonucleotides containing 8-aminopurines are known by those skilled in the art. However, natural oligonucleotides containing 8-aminopurines cannot be directly used for the specific binding of double-stranded DNA sequences, since the modified bases are purines that are in the target sequence and not in the Hoogsteen strand used for specific recognition of double-stranded DNA in usual triplex strategies.

We describe the binding properties of hairpins carrying 8-aminopurines, such as for example but not limited to, 8-aminoadenine, 8-aminopurine and 8-aminohypoxanthine connected head-to-head to the Hoogsteen pyrimidine strand (FIG. 14). Hairpins carrying 8-aminopurines form stable Hoogsteen parallel-stranded structures. We show that these modified hairpins of this invention bind to the Watson-Crick pyrimidine strand via a triple helix with greater affinity than hairpins containing only natural bases, especially in neutral conditions. The effect of pH, salt concentration and loop structure on triplex stability are also analyzed herein. Moreover, parallel-stranded hairpins of this invention are shown to form triplexes with a base interruption in the polypyrimidine target sequence. The increased stability of the triple helix at neutral conditions and the possibility to cope with the interruptions in the polypyrimidine target sequences create new applications based on triple helix formation such as structural studies, DNA-based diagnostic tools, antigene and antisense therapies.

Methods

Molecular Dynamics (MD) Simulations.

We analyzed the stability of a 11-mer parallel DNA duplex with generally the same content of d(G·C) and d(A·T) pairs—d(5′-GAAGGAGGAGA-3′)d(5′-CTTC-CTCCTCT-3′) (SEQ ID NO: 1, SEQ ID NO: 2)—in water at room temperature when the base pairing corresponds to both rWC and H motifs. Two and three starting models were considered for H and rWC duplexes, respectively (Table 1). The two starting models for H duplex were obtained by removing the pyrimidine Watson-Crick strand of a A- and B-type triplex (simulations

TABLE 1
Summary of Starting Structures and Simulation Times Used for MD
Analysis of Parallel-stranded Duplexes^{a}

| | | length of |
|---|---|---|
| pairing scheme | starting structure | simuln (ns) |
| Hoogsteen | modeled from B-type triplex^{e} | 5 |
| Hoogsteen | modeled from A-type triplex^{e} | 5 |
| rev Watson-Crick | modeled from NMR data^{f} | 2^{b} |
| rev Watson-Crick | modeled from theoretical model^{g} | 1^{b} |
| rev Watson-Crick^{c} | from an MD model^{g} | 5 |
| Watson-Crick^{d} | from canonical model^{h} | 5 |

^{a}In all cases structures were modeled by substitution of the original sequence by the studied one prior to the optimization and equilibration process. In the case of rWC pairs the structures were modeled to show a double hydrogen-bond scheme.
^{b}Simulation was stopped at that time because the structure of the helix was severely distorted.
^{c}The original rWC d(A · T) Pattabiraman structure was equilibrated for 1 ns using MD; the sequence was then modified to that of interest, then reoptimized, and reequilibrated.
^{d}Antiparallel duplex.
^{e}Shields G,; Laughton, C. A.; Orozco, M. J. Am. Chem. Soc. 1997, 117, 7463; Soliva, R.; Laughton, C. A.; Luque, F. J.; Orozco, M. J. Am. Chem. Soc. 1998, 120, 11226; Shields, G.; Laughton, C. A.; Orozco, M. J. Am. Chem. Soc. 1998, 120, 5895.
^{f}Yang, X. L.; Sugiyama, H.; Ikeda, S.; Saito, I.; Wang, A. H. Biophys. J. 1998, 75, 1163.
^{g}Pattabiraman, N. Biopolymers 1986, 25, 1603.
^{h}Arnott S.; Hukins, D. W. L. Biochem. Biophys. Res. Commun. 1972, 47, 1504. All publications cited in this table are incorporated by reference herein.

H_{A} and H_{B}). The three starting models for rWC duplex correspond to (i) the NMR model, (ii) the canonical model reported by Pattabiraman and (iii) an equilibrated MD rWC d(A·T) duplex (see Table 1). These starting structures lead to simulations rWC₁ rWC₂, and rWC₃, respectively. For comparison purposes an antiparallel B-type duplex of the same sequence was generated using canonical structural parameters. In all cases the duplex was immersed in a box containing 2200-2700 water molecules and sodium ions were added to neutralize the system. Based on previous results Hoogsteen cytosines were protonated. The hydrated duplexes were then optimized, thermalized, and equilibrated for 130 ps. All the systems were then subjected to 1-5 ns (nanosecond) of unrestrained MD simulation at constant pressure (1 atm) and temperature (298 degrees K.) using periodic boundary conditions and the particle-mesh Ewald method known by those skilled in the art to account for long-range electrostatic effects. SHAKE (J. Comput. Phys. 1977, Vol. 23, pg. 327) was used to maintain all the bonds at their equilibrium distances, which allowed us the use of a 2 fs time step for integration. AMBER-98/TIP3P (J. Am. Chem. Soc., 1995, Vol. 117, page 5179) and previously developed parameters for protonated cytosines and 8-aminopurines were used.

Geometrical analysis of the trajectories was performed using exclusively the central 9-mer duplex. The two trajectories of the H-based duplexes were averaged to obtain a better (10 ns) representation of the duplex. Analysis of possible molecular interactions of DNA was carried out using the CMIP program (CMIP computer program, made available by the University of Barcelona, Barcelona, Spain), and structural analysis of the trajectories performed.

Free Energy Calculations.

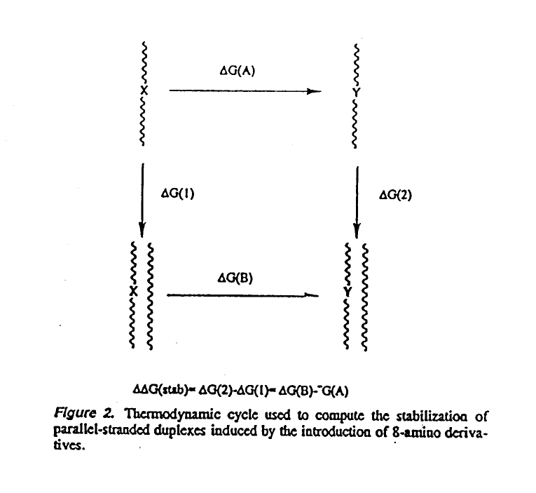

Thermodynamic integration technique coupled to molecular dynamics simulations (MD/TI) was used to analyze the effect of replacing 2′-deoxyadenosine, 2′-deoxyguanosine, and 2′-deoxyinosine by their 8-amino derivatives on the stability of the d(5′-GAAGGAGGAGA-3′)d(5′-CTTCCTCCTCT-3′) (SEQ ID NO: 1, SEQ ID NO: 2) parallel-stranded duplex. In this embodiment of the present invention, mutations were performed between 8-amino-2′-deoxyadenosine and 2′-deoxyadenosine (8AA→A), 8 amino-2′-deoxguanosine and 2′-deoxyguanosine (8AG→G), and 8-amino-2′-deoxyinosine and 2′-deoxyinosine (8AI→I) in both duplex and single-stranded oligonucleotides. The change in stabilization free energy due to the 8AX→X mutation is determined using standard thermodynamic cycles as known by those skilled in the art. (FIG. 2).

MD/TI simulations were done considering only the H duplex due to the instability of the rWC duplex. The starting system in these calculations was defined as that obtained at the third nanosecond of the MD simulation duplex corresponding to the B trajectory of the H duplex. The 8-amino derivatives were then modeled at position 5 (8AG and 8AI) or 6 (8AA) of the purine strand, and the resulting structures were further equilibrated for 0.5 ns to avoid any bias in the calculations. Two additional simulations were performed considering the d(G·C)/d(I·C) pair at position 5 shows a wobble neutral pairing, d(G·C)_{w}/d(I·C)_{w}, instead of the normal protonated pair, d(G·C)⁺/d(I·C)⁺. In this case one extra sodium ion was added to the modeled system, which was then further equilibrated for ins. The single strands were modeled as 5-mer oligonucleotides of sequences 5′-AGGAG-3′, 5′-AG/AG-3′, and 5′-GGAGG-3′.

Mutations were performed using 21 double-wide windows of 10 and 20 ps each, leading to trajectories of 420 or 820 ps. Free energy estimates were obtained using the first and second halves of each window, which allows two independent estimates of the free energy change for every simulation. The values presented here correspond then to the average of four independent estimates, for estimating the statistical uncertainty of the averages. All other technical details of MD/TI simulations are identical to those of MD calculations. Simulations presented here correspond to more than 30 ns of unrestrained MD simulations of 11-mer H duplexes in water.

All MD and MD/TI simulations were carried out using the AMBER-5.1 computer program. All simulations were done in the supercomputers of the Centre de Supercomputacio de Catalunya (CESCA).

Preparation of Oligomers Containing 8-Aminopurines.

Oligonucleotides were prepared on an automatic DNA synthesizer using standard and reversed 2-cyanoethyl phosphoramidites and the corresponding phosphoramidites of the 8-aminopurines. The phosphoramidite of protected 8-amino-2′-deoxyinosine was dissolved in dry dichloromethane to make a 0.1 M solution. The rest of the phosphoramidites were dissolved in dry acetonitrile (0.1 M solution). The phosphoramidite of the hexaethylene glycol linker was obtained from commercial sources known in the art. The preparation of 3′-3′ linked hairpins (R-22 (SEQ ID NO: 1, SEQ ID NO: 2) R-22A (SEQ ID NO: 3, SEQ ID NO: 2) R-22G (SEQ ID NO: 4, SEQ ID NO: 2) and R-22G (SEQ ID NO: 5, SEQ ID NO: 2) was performed in three parts: First was the preparation of the pyrimidine part, using reversed C and T phosphoramidites and reversed C support (linked to the support through the 5′ end). Next, after the assembly of the pyrimidine part, a hexaethylene glycol linker was added using a commercially available phosphoramidite known by those skilled in the art. Finally, the purine part carrying the modified 8-aminopurines was assembled using standard phosphoramidites for the natural bases and the 8-aminopurine phosphoramidites. For the preparation of 5′<5′ linked hairpins B-22 (SEQ ID NO: 1, SEQ ID NO: 2) B-22A (SEQ ID NO: 3, SEQ ID NO: 2) B-22G (SEQ ID NO: 4, SEQ ID NO: 2) B-22AG (SEQ ID NO: 5, SEQ ID NO: 2) B-AT (SEQ ID NO: 6, SEQ ID NO: 7), and B-22A control (SEQ ID NO: 3, SEQ ID NO: 8) a similar approach was used. In this case, the purine part was assembled first, followed by the hexaethylene glycol linker. The pyrimidine part was the last part to be assembled using reversed phosphoramidites. Complementary oligonucleotides containing natural bases were also prepared using commercially available chemicals and following standard protocols known by those skilled in the art. After the assembly of the sequences, oligonucleotide supports were treated with 32% aqueous ammonia at 55° C. for 16 h (hour) except for oligonucleotides having 8-aminopurine. In this case a 0.1 M 2-mercaptoethanol solution in 32% aqueous ammonia was used and the treatment was extended to 24 h (hour) at 55° C. (Centigrade). Ammonia solutions were concentrated to dryness and the products were purified by reverse-phase HPLC. Oligonucleotides were synthesized on a 0.2 μmol scale and with the last DMT group at the 5′ end (DMT on protocol) to help reverse-phase purification. All purified products presented a major peak, which was collected. Yields (OD units at 260 nm after HPLC purification, 0.2 μmol) were between 6 and 10 OD. HPLC conditions: HPLC solutions are as follows. Solvent A, 5% ACN in 100 mM triethylammonium acetate (pH 6.5); and solvent B, 70% ACN in 100 mM triethylammonium acetate pH 6.5. Columns: PRP-1 (Hamilton), 250×10 mm. Flow rate 3 mL/min. A 30 min linear gradient from 10 to 80% B (DMT on), or a 30 min linear gradient from 0 to 50% B (DMT off).

Melting Experiments.

Melting experiments were performed as follows: Solutions of the hairpins and duplexes were dissolved in 1 M NaCl, 100 mM phosphate/citric acid buffer. The solutions were heated to 90° C., and then allowed to cool slowly to room temperature, and then samples were kept in the refrigerator overnight. UV absorption spectra and melting experiments (absorbance vs temperature) were recorded in 1 cm path length cells using a spectrophotometer, which has a temperature controller with a programmed temperature increase of 0.5° C./min. Melts were run on duplex concentration of 3-4 μM at 260 nm.

Circular Dichroism (CD).

Oligonucleotides were dissolved in 100 mM phosphate buffer pH 6.0, 50 mM sodium chloride, and 10 mM magnesium chloride. The equimolar concentration of each strand was 4-5 μM. The solutions were heated at 90° C., allowed to come slowly to room temperature, and stored at 4° C. until CD measurement was performed. The CD spectra were recorded on a Jasco J-720 spectropolarimeter attached to a Neslab RP-100 circulating water bath in 1 cm path length quartz cylindric cells. Spectra were recorded at room temperature using a 10 nm/min scan speed, a spectral bandwidth of 1 nm, and a time constant of 4 s. CD melting curves were recorded at 280 nm using a heating rate of 20° C./h and a scan speed of 100 nm/ min. All the spectra were subtracted with the buffer blank, normalized to facilitate comparisons, and noise-reduced using Microcal Origin 5.0 software.

NMR Spectroscopy. A sample of the oligonucleotide d(3′-AGNGGNGGAAG-5′-(EG)₆-5′-CTTCCTCCTCT-3′) (N=8-amino-A) (SEQ ID NO: 3, SEQ ID NO. 2) for NMR experiments was prepared in 250 pL of 9:1 H₂O/ D₂O, 25 mM sodium phosphate buffer, and 100 mM NaCl. The pH was adjusted by adding small amounts of concentrated HCl. The final oligonucleotide concentration was around 1 mM. Spectra were acquired in a Bruker AMX spectrometer operating at 600 MHz, and processed with the UXNMR software. Water suppression was performed by using a jump-and-return pulse sequence with a null excitation in the water signal. All experiments were performed at 5° C.

Molecular Dynamics Simulations.

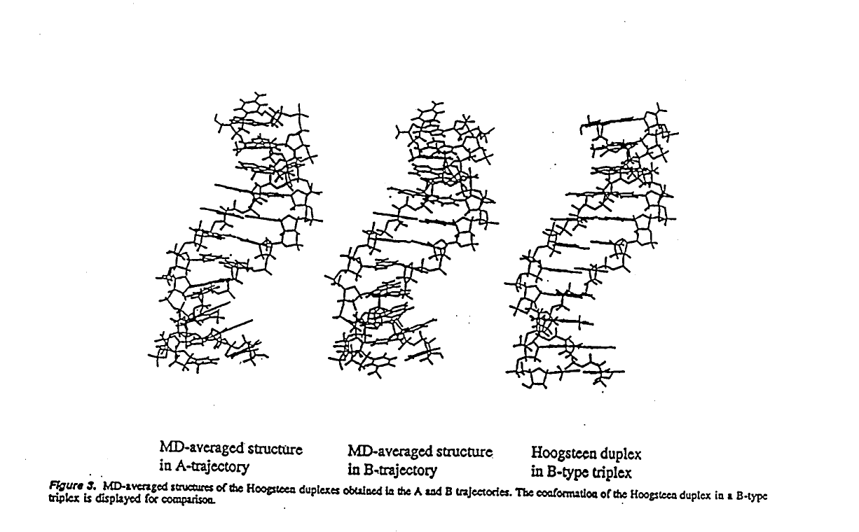

MD simulations of H duplexes show stable trajectories along the 5 ns simulation time (FIG. 3), as noted in the average root-mean-square deviation (rmsd) between the trajectories and the respective MD-averaged conformations (1.4 and 1.5 Å for simulations H_{A} and H_{B}, respectively). The only noticeable distortions are a slight bend at the d(G·C) end and the existence of partial fraying events at the d(A·T) end. Similar features occured in the control antiparallel helix. It is also worth noting that the existence of two consecutive protonated pairs d(G·C)⁺ did not introduce large structural alterations in the helix.

The two simulations, which started from different H-based duplex models, are reasonably converged and sample similar regions of the conformational space. This is noted in the rmsd between each trajectory and the MD-averaged conformation of the other: 1.9 Å (B trajectory with respect to the average structure in simulation H_{A}) and 2.1 Å (A trajectory with respect to the average structure in simulation H_{B}). Both trajectories sample conformational regions close to those typical of Hoogsteen strands in a triplex DNA (FIG. 3). It will be appreciated by those persons skilled in the art that the MD simulations suggest that the structure of the Hoogsteen strands of a triplex is not largely distorted when the pyrimidine Watson-Crick strand is removed. Thus, the rmsd between the two trajectories and the starting model in simulation H_{B} (taken directly from a B-type triplex DNA) is 1.4 and 1.8 Å in simulations H_{B} and H_{A}, respectively. The rmsd is slightly larger with respect to the Hoogsteen strands of the starting model in simulation H_{A} (an A-type triplex): 2.0 Å (H_{A}) and 2.1 Å (H_{B}).

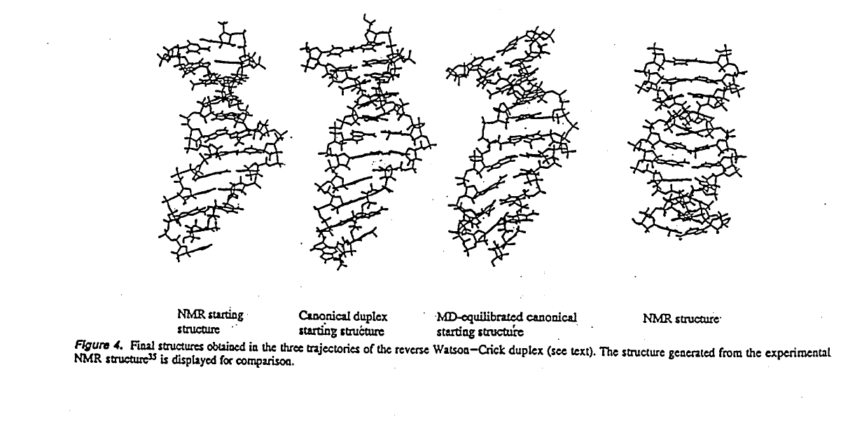

In contrast to these results, the simulations of rWC duplexes starting from the high-resolution NMR or the canonical model (simulations rWC₁ and rWC₂) diverge very quickly. All the efforts to reinforce the equilibration of the system and the pairing between bases fail to provide stable structures (rmsd from canonical structure 3.4-3.9 Å at the end of the simulations). Beside the fact that many interstrand hydrogen bonds and stacking interactions are preserved along the simulation, the geometries are heavily distorted in less than 1 ns (see FIG. 4), and the helical nature of the structures is then completely lost. The third simulation (rWC₃), which started from a model derived from a previously 1 ns equilibrated trajectory of a d(A·T) rWC duplex, was stable for a longer period, but the helix was also largely distorted (rnsd 3.2 Å) after the 5 ns simulation time (FIG. 4). While not wishing to be bound by a particular theory, analysis of the trajectories suggests that the amino repulsion between G and C is the main factor that causes the helix destabilization, despite our efforts to reduce the amino repulsion by promoting a wobble d(G·C) pairing.

The MD simulations suggest that the rWC duplex is not stable. On the contrary, the H-based conformation seems stable during all the simulation time. Therefore, the results support the existence of H-based motifs for parallel-stranded duplexes in DNAs with similar population of d(A·T) and d(G·C) pairs, and that the rWC helix is not stable when there is a high content of d(G·C) pairs.

The stability of the H-based simulations allows analysis of the structure of a H-based parallel-stranded duplex. As noted above, the helix is similar to the structure of Hoogsteen strands in a DNA triplex. The average twist is 31°, and the rise is 3.4 Å. The bases are generally perpendicular to the helix axis. The sugars are in the South and South-East regions, having an average phase angle of 124°, as found experimentally for rWC parallel-stranded duplexes and triplexes. There is a narrow groove (denoted “minor” in the following) corresponding to the minor part of the major groove in DNA triplexes, and a wide groove (denoted here “major”) corresponding to both the minor groove and the major part of the major groove of a DNA triplex (FIG. 3). The shortest P-P average distance along the two grooves is around 9 (±0.6) and 25 (±2) Å for the “minor” and “major” grooves. There are then major differences with rWC duplexes, where two equivalent grooves were found.

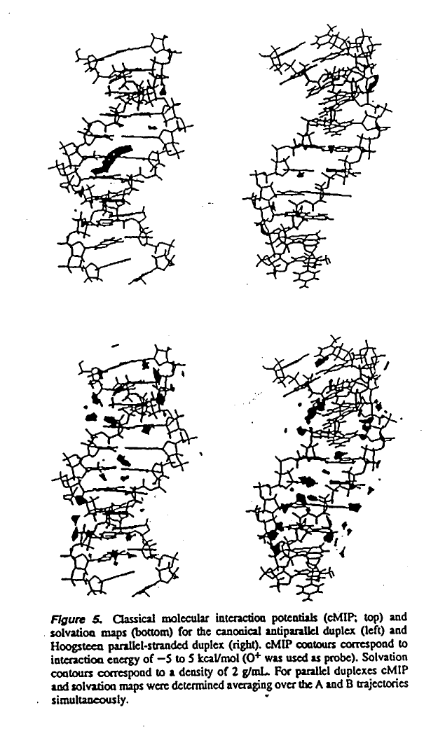

The classical molecular interaction potential maps (CMIP; FIG. 5) allowed us to trace the regions where the DNA has a strong propensity to interact with small cationic probes. As expected from our previous studies on DNA triplexes, the “minor” groove is the most active region for interactions. The ability of the H duplex to interact the cationic probes is not different from that of a B-type antiparallel duplex with the same sequence, despite the fact that all Hoogsteen cytosines are protonated in the H duplex. It is clear that the short P--P distance in H duplexes creates a strong negative potential in the vicinities of the Hoogsteen cytosines, thus screening their positive charge.

The H duplex is very well hydrated, as shown in the solvation contours represented in FIG. 5. The largest apparent density of water is found in the minor groove, which is wide enough to allow the insertion of a chain of ordered waters. There are also regions of large (more than 2 g/mL) water density in the vicinities of the phosphate groups in the major groove. Interestingly, the apparent water densities around the H duplex and the reference antiparallel helix are very similar, thus confirming the findings obtained from CMIP calculations.

The antiparallel H duplex is a new structure which shares many characteristics with DNA triplexes, but that also exhibits a series of unique molecular recognition characteristics derived mainly from the existence of two very different grooves.

Free Energy Calculations.

The design, synthesis, and evaluation of a series of 8-amino derivatives of purine bases are known by those skilled in the art. These molecules strongly stabilize the DNA triplex, which was related, among other factors, to an extra hydrogen bond between the 8-amino group of the purine and the carbonyl group of Hoogsteen cytosines or thymines. We also found that the 8-amino group promotes a strong destabilization of the Watson-Crick pairing, at least for d(G·C) and d(I·C) pairs. Accordingly, we could expect that the presence of 8-amino groups should destabilize the rWC duplex, increasing the stability of the H duplex. It is worth noting that the stability of the H duplex is crucial for the use of parallel-stranded duplexes as templates for triplex formation. MD/TI calculations were performed only in the H duplex because the instability of the rWC duplex precludes any TI calculation. As found in previous simulations for related systems, the mutation profiles are smooth, without any apparent discontinuity, which could signal the existence of hysteresis. The standard errors in free energy estimates are 0.2-0.3 kcal/mol, thus indicating a good convergence in the results (Table 2).

The H duplex is stabilized by around 2.7 kcal/mol by the A→8AA mutation (Table 2), a value similar to that found previously using less rigorous simulation protocols for poly d(A-T-T) triplex. The mutation G→8AG in a d(G-C)⁺ motif increases the stability of the H duplex by around 1 kcal/mol.

TABLE 2
MD/TI Estimates of Stabilization (ΔΔG_{stab} and Standard Errors in
kcal/mol) of Parallel-Stranded Duplexes induced by 8-Amino Derivatives^{a}

| mutation | complementary pyrimidine | ΔΔG_{stab} kcal/mol |
|---|---|---|
| G→8AG | C+ | −1.4 ± 0.2 |
| G→8AG | C | −3.1 ± 0.3 |
| I→8AI | C+ | −0.9 ± 0.3 |
| I→8AI | C | −3.2 ± 0.3 |
| A→8AA | T | −2.7 ± 0.3 |

^{a}For d(G · C) and d(I · C) motifs the simulation was performed considering two ionization states of the Hoogsteen cytosine. Calculations were carried out always using the sequence d(GAAGXAGGAG), (SEQ ID NO: 10) where X is the base which is mutated.

(Table 2), while the I→8AI mutation in the d(I-C)⁺ motif increases the stability by around 1.4 kcal/mol (Table 2). These two latter values also agree with previous estimates in DNA triplexes.

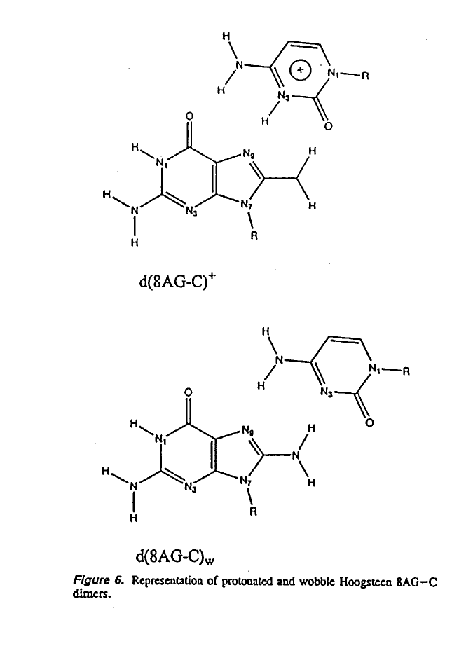

Results noted herein clearly point out a strong stabilization of the H duplex upon introduction of 8-aminopurines and suggest that these molecules can help stabilize hairpins based on the parallel H duplex. We were, however, concerned by the fact that the G→8AG mutation stabilizes the H duplex less than the A→8AA mutation, since this finding, which agrees with previous calculations in triplexes, does not agree with melting experiments on H hairpins (see below). While not wishing to be bound by a particular theory, this suggests that when 8 AG (or 8AI) is present, the Hoogsteen recognition might not necessarily be the d(8AG·C)⁺ motif, but can be a wobble pair d(8AG·C)_{w} (see FIG. 6). Because the d(G/I·C)⁺→d(8AG/8AI·C)_{w} mutation is technically very difficult owing to the annihilation of a net charge, we investigated by means of indirect evidence the potential role of d(8AG-C)_{w} motifs by doing the mutations G→8AG and I→8AI in the presence of a neutral cytosine in the complementary Hoogsteen position (the rest of the Hoogsteen cytosines were protonated). The results (see Table 2) suggest that the presence of 8-amino derivatives strongly stabilizes (3.1 and 3.2 kcal/mol for I and G, respectively) the wobble pairing. Note that this free energy difference is 0.5 kcal/mol larger than that found in the A→8AA mutation and more that 2 kcal/mol larger than the stabilization due to the same mutation when the Hoogsteen cytosine is protonated. According to these results, it is believed that the presence of 8AG and 8AI favors the existence of neutral Hoogsteen motifs instead of the protonated ones (see below). This could be due to the fact that the 8-amino is a hydrogen-bond donor which interacts better with a neutral molecule than with a cation.

Structure of the Oligonucleotide Derivatives.

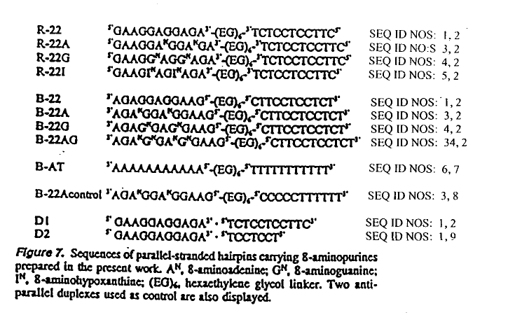

To check MD and MD/TI-derived hypothesis, several parallel-stranded DNA hairpins carrying 8-aminoadenine (8AA=A^{N}), 8-aminopurine (8AG=G^{N}), and 8-aminohypoxanthine N (8AI=I^{N}) were prepared. The sequences of the oligonucleotides are shown in FIG. 7.

The first group of oligomers are parallel-stranded hairpins connected through their 3′ ends with an hexaethylene glycol linker [(EG)₆]. Two adenines are substituted by two 8-aminoadenines (A^{N}) in the oligonucleotide R-22A (SEQ ID NO: 3, SEQ ID NO: 2) the oligonucleotide R-22G (SEQ ID NO: 4, SEQ ID NO: 2) two guanines are substituted by two 8-aminoguanines (G^{N}), and in the oligonucleotide R-221 (SEQ ID NO: 5, SEQ ID NO: 2) two guanines are substituted by two 8-aminohypoxanthines (I^{N}). The oligonucleotide (R-22) (SEQ ID NO: 1, SEQ ID NO: 2) contains only the natural bases without modification.

TABLE 3
Melting Temperatures^{a} (° C.) for the Parallel-Stranded Hairpins Having 3′-
3′ Linkages

| hairpin | pH 4.6 | pH 5.5 | pH 6.0 | pH 6.5 | pH 7.0 |
|---|---|---|---|---|---|
| R-22*1 | 46 | 34 | 25 | | |
| R-22A*2 | 64 | 50 | 43 | 28 | |
| R-22G*3 | 68 | 55 | 50 | 40 | 39 |
| R-22I*4 | 52 | 42 | 34 | 25 | 23 |

^{a}InI M NaCl, 100 mM sodium phosphate/citric acid buffer.
*1(SEQ ID NO: 1, SEQ ID NO: 2)
*2(SEQ ID NO: 3, SEQ ID NO: 2)
*3(SEQ ID NO: 4, SEQ ID NO: 2)
*4(SEQ ID NO: 5, SEQ ID NO: 2)

The second group of oligomers B-22 (SEQ ID NO: 1, SEQ ID NO: 2) B-22A (SEQ ID NO: 3, SEQ ID NO: 2) B-22G (SEQ ID NO: 4, SEQ ID NO: 2) is similar in composition to those in the previous oligomers, but the polypurine and the polypyrimidine parts are connected through their 5′ ends with an hexaethylene glycol linker [(EG)₆]. In addition, an oligomer having two 8-aminoguanines and two 8-aminoadenines was prepared (B-22AG) (SEQ ID NO: 34, SEQ ID NO: 2) to test whether the stabilizing properties of both 8-aminopurines are additive. A parallel-stranded hairpin that has only d(A·T) base pairs (B·AT) (SEQ ID NO: 6, SEQ ID NO: 7) was prepared. Finally, a control hairpin (B-22A control) (SEQ ID NO: 3, SEQ ID NO: 8) with the same purine sequence as B22A (SEQ ID NO: 3, SEQ ID NO: 2) but a noncomplementary pyrimidine sequence was also prepared.

Oligonucleotide sequences containing 8-aminopurines were prepared using phosphoramidite chemistry on an automatic DNA synthesizer. The parallel-stranded oligomers were prepared using protocols known by those skilled in the art. The phosphoramidites of 8-aminoadenine, 8-aminopurine, and 8-aminohypoxanthine were prepared using protocols known by those skilled in the art.

Melting Experiments.

The relative stability of parallel-stranded hairpins was measured spectrophotometrically at different pHs (pH 4.6-7.0). In most cases one single transition was observed with an hyperchromicity around 15% at acidic pH and 10% at neutral pH that was assigned to the denaturation of the parallel-stranded hairpin. In Table 3, melting temperatures of the hairpins having 3′<3′ linkages are shown.

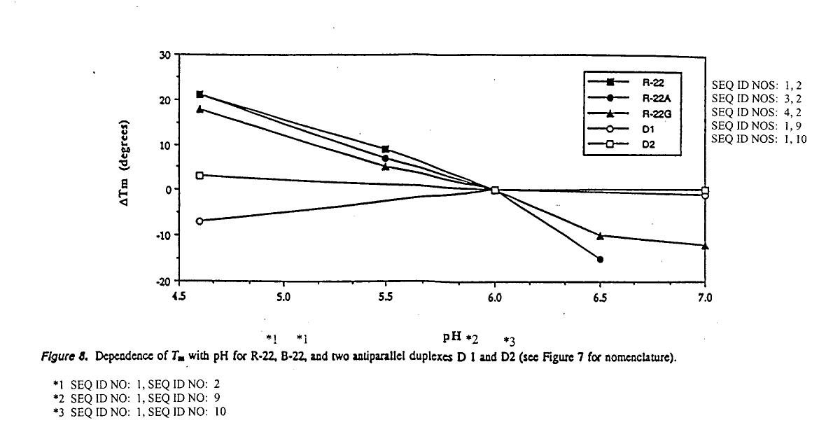

When the hairpin is formed by natural bases (R-22) (SEQ ID NO: 1, SEQ ID NO: 2), a clear transition is observed at pH 4.6 and pH 6.0 but no transition was observed at pH higher than 6.0. Melting temperatures are pH-dependent, and at lower pH melting temperatures are higher than at pH 7.0. These results are consistent with a Hoogsteen base pairing in which C has to be protonated (i.e., an H-type duplex is supported). This profile of pH dependence cannot be explained for a reverse Watson-Crick parallel duplex, and it is also inconsistent with the existence of short antiparallel duplexes (like a 7-mer duplex d(-AGGAGGA-)·d(-TCCTCCT-), which could be formed with the central part of sequence. To verify the latter point we synthesized and measured the melting temperatures at pH 4.5, 6.0, and 7.0 of two antiparallel duplexes of sequences d(GAAGGAGGAGA)·d(TCTCCTCCTTC) (SEQ ID NO: 1, SEQ ID NO: 2) (DI) and d(GAAGGAGGAGA)·d(TCCTCCT) (SEQ ID NO:1, SEQ ID NO: 9) (D2). The profiles of pH dependence with the temperature found for both antiparallel duplexes are compared in FIG. 8 with those found for R-22 (SEQ ID NO: 1, SEQ ID NO: 2) and B-22 (SEQ ID NO: 1, SEQ ID NO: 2). It is clear that the profiles strongly support that the antiparallel duplex is not significantly populated.

The substitution of two A's by two 8AAs stabilizes the parallel-stranded structure as seen by the higher melting temperatures at pH 4.6 and 6.0 (ΔTₘ 16-18° C.) and the observation of a transition at pH 6.5. The substitution of two G's by two 8AGs raises the melting temperatures of the hairpins even higher. The differences in melting temperatures with respect to B-22 (SEQ ID NO: 1, SEQ ID NO: 2) are between 21 and 25° C. It is also possible to observe a transition at about pH 7.0 and 6.5. The substitution of two G's by two 8AIs stabilizes the parallel-stranded structure, but this stabilization is of small intensity (ΔTₘ 6-9° C. at pH 4.6-6.0). The melting temperatures of hairpins having 8AG and 8AI are not decreasing so quickly at neutral pH. This indicates that these hairpins are not as dependent as the other hairpins to protonation of C probably due to the extra hydrogen bond between the 8-amino group of the 8-aminopurines and the 2-keto group of C.

As noted herein, in addition to the hairpins linked by 3′<3′ bonds (R-22 (SEQ ID NO: 1, SEQ ID NO: 2) derivatives) we prepared hairpins linked by 5′<5′ bonds (B-22 (SEQ ID NO: 1, SEQ ID NO: 2) derivatives). Table 4 shows the melting temperatures of these hairpins at different pHs.

TABLE 4
pMelting Temperatures^{a} (° C.) for the Parallel-Stranded Hairpins Having 5′-
5′ Linkages

| | | | | | |
|---|---|---|---|---|---|
| hairpin | pH 4.6 | pH 5.5 | pH 6.0 | pH 6.5 | pH 70 |

| | | | | | |
|---|---|---|---|---|---|
| B-22*1 | 57 | 35 | 25 | | |
| B-22A*2 | 61 | 47 | 38 | 23 | |
| B-22G*3 | 65 | 54 | 44 | 30 | 21 |
| B-22AG*4 | 72 | 62 | 52 | 43 | 39 |

^{a}InI M NaCl, 100 mM sodium phosphate/citric acid buffer.
*1SEQ ID NO: 1, SEQ ID NO: 2
*2SEQ ID NO: 3, SEQ ID NO: 2
*3SEQ ID NO: 4, SEQ ID NO: 2
*4SEQ ID NO: 34, SEQ ID NO: 2

Results are similar to that described herein with hairpins having 3′-3′ linkages. Substitution of A or G by the corresponding 8-aminopurine derivative induces a strong stabilization of the hairpin seen as a higher Tₘ at acidic pH and the observation of transitions at neutral pH that are not possible to observe with hairpins having only natural bases. It is important to notice also that the addition of both 8AA and 8AG in the same oligonucleotide (B-22AG) (SEQ ID NO: 34, SEQ ID NO: 2)has additive effects. For example, at pH 6.0, the presence of two 8AAs gives an increase on the Tₘ of 13° C., two AGs give an increase of 19° C. and the addition of both two 8AAs and two 8AGs gives an increase of 27° C. The low dependence of melting temperatures with the pH found for values close to pH 7.0 for hairpins having 8AG (R-22G) (SEQ ID NO: 4, SEQ ID NO: 2) and 8AI (R-22G (SEQ ID NO: 5, SEQ ID NO: 2) is observed for hairpin B-22AG (SEQ ID NO: 34, SEQ ID NO: 2) but not for hairpin B-22G (SEQ ID NO: 4, SEQ ID NO: 2). Parallel hairpins containing only A-T pairs (B-AT (SEQ ID NO: 6, SEQ ID NO: 7)) had the same melting temperature (Tₘ=42° C.) from about pH=5.5 to 7.0. Control hairpin (B-22A control (SEQ ID NO: 3, SEQ ID NO: 8)) had no transition at any pH.

All the melting experiments described in Tables 3 and 4 were performed at about 1 M NaCl, as described under the methods set forth herein. In addition, we have performed melting experiments from about 0 to 1 M NaCl. Melting temperatures remain unchanged within 1 degree error, in agreement with previous results regarding salt effects in Hoogsteen pairing.

There is excellent agreement between MD/TI calculations derived from the assumption of an H-type parallel duplex and experimental measures. The large stabilization found theoretically for the amino groups is also detected experimentally in increases in Tₘ of almost 10° C. per substitution. Interestingly, the greater stability obtained for the G→8AG mutation compared with that obtained by the A →8AA mutation and the smaller dependence on pH of the stability of duplexes containing 8AG suggest that neutral wobble pairing might play a key role in parallel duplexes containing d(8AG·C) pairs. Finally, the small stabilization obtained for the G→8AI mutation is the result of the balance between the stabilization of the H-duplex induced by the I→8AI mutation and the destabilization induced by the G→I change.

The 8-amino group destabilizes the Watson-Crick pairing for G and I and is expected then to destabilize the reverse Watson-Crick pairing. Accordingly, the stabilization in the duplex structure found experimentally can be understood only considering that the hairpins studied here have a Hoogsteen and not a reverse Watson-Crick secondary structure. Note also that the change in stability of the duplex induced by the G→8AG or A→8AA substitutions also argue strongly against the existence of significant amounts of a 7-mer antiparallel duplex. Thus, the changes of two G's (positions 5 and 8) by two 8AGs lead to a decrease of 7° C. in Tₘ for the two antiparallel duplexes used as controls d(GAAGGAGGAGA) ·d (TCTCCTCCTTC) (SEQ ID NO: 1, SEQ ID NO: 2) and d(GAAGGAGGAGA) ·d(TCCTCCT), (SEQ ID NO: 1, SEQ ID NO: 9)while for R-22 (SEQ ID NO: 1, SEQ ID NO: 2) and B-22 (SEQ ID NO: 1, SEQ ID NO: 2) the same changes induced an increase of more than 21° C. in Tₘ.

Circular Dichroism.

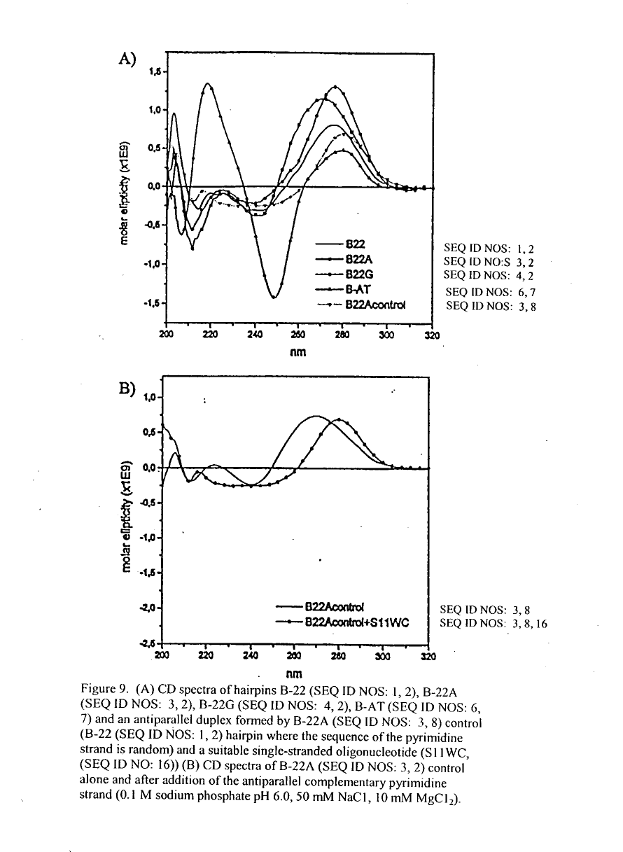

To obtain information on the structure of the hairpins, circular dichroism (CD) spectra were measured. FIG. 9A shows the CD spectra of hairpins B-22, (SEQ ID NO: 1, SEQ ID NO: 2), B-22A (SEQ ID NO: 3, SEQ ID NO: 2) and B-22G (SEQ ID NO:4, SEQ ID NO: 2) and the parallel-stranded hairpin with d(A·T) base pairs (B-AT (SEQ ID NO: 6, SEQ ID NO: 7)). As an additional control, we introduced a modified B22 hairpin (B-22A control(SEQ ID NO: 3, SEQ ID NO: 8), where the sequence of the pyrimidine strand is random, to guarantee that no parallel duplex can be formed. This later oligonucleotide was paired with the corresponding 1-mer oligonucleotide complementary to the WC purine strand (S11WC) (SEQ ID NO:16). As noted in FIG. 9B, B-22A control (SEQ ID NO: 3, SEQ ID NO: 8) does not have structure, but it generates an antiparallel duplex if a suitable single-stranded oligonucleotidic strand (S11WC) (SEQ ID NO: 16) is added (B-22A control+S11WC) (SEQ ID NO: 3, SEQ ID NO: 8, SEQ ID NO: 16).

The shapes of the CD spectra (see FIG. 9A) of hairpins B-22, (SEQ ID NO: 1, SEQ ID NO: 2) B-22A (SEQ ID NO: 3, SEQ ID NO: 2) and B-22G (SEQ ID NO: 4, SEQ ID NO: 2) are similar, and clearly differ from B-AT (SEQ ID NO: 6, SEQ ID NO: 7) and from the antiparallel duplex (B-22A control+S11WC (SEQ ID NO: 3, SEQ ID NO: 8, SEQ ID NO: 16); see also FIG. 9B). The CD spectra of the hairpin B-AT (SEQ ID NO: 6, SEQ ID NO: 7) has a strong minimum at 248 nm, a smaller minimum at 206 nm, and two maxima at 218 and 280 nm. This spectrum is similar to that known in the art for A-T rich parallel-stranded DNA that is considered a model for reverse Watson-Crick pairing. The CD spectra of B-22, (SEQ ID NO: 1, SEQ ID NO: 2) B-22A (SEQ ID NO: 3, SEQ ID NO: 2) and B-22G (SEQ ID NO:4, SEQ ID NO: 2) have a strong maximum between 270 and 290 nm and two minima: one at 242 nm and a second, more intense minima at around 212 nm. The minimum around 212 and the maximum around 280 are more intense in the hairpins containing 8-aminopurines (B-22A (SEQ ID NO: 3, SEQ ID NO: 2) and B-22G(SEQ ID NO: 4, SEQ ID NO: 2)). This type of spectra is characteristic of DNA triplexes. In summary, CD spectra demonstrate that the hairpins of this invention, which contain a mixture of A(8AA)-T and G(8AG/8AI)-C steps have a Hoogsteen-type structure and are not reverse Watson-Crick parallel or Watson-Crick antiparallel duplexes.

NMR Spectra.

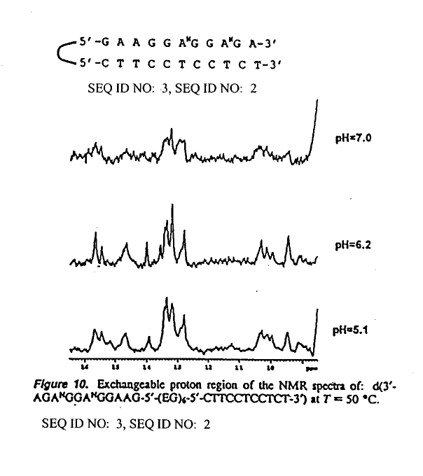

The imino region of one-dimensional ¹H NMR spectra of the DNA hairpin d(3′-AGA^{N}GGA^{N}GGAAG-5′-(EG)₆-5′-CTTCCTCCTCT-3′) (SEQ ID NO: 3, SEQ ID NO: 2) at three different pHs is shown in FIG. 10. Unfortunately, the broad signals observed (due probably to the formation of Hoogsteen parallel inter-molecular duplexes at the concentration of NMR experiment) prevented the acquisition of high-quality two-dimensional spectra, and, therefore, the sequential assignments could not be done. However, the presence of imino signals between 14.5 and 16.0 ppm clearly indicates that some cytosines are protonated. Also, the signals around 10 ppm correspond to amino protons of cytosines forming Hoogsteen base pairs. Most probably, the resonances around 13 ppm are due to imino protons of Hoogsteen thymines. Since the chemical shifts of the exchangeable protons in reversed Watson-Crick base pairs are very similar to those observed in canonical antiparallel duplexes, this kind of base pairing can be ruled out. Finally, it is worth noting that most of the features of the exchangeable proton spectra can be still observed at neutral pH, suggesting a notable stability of the parallel duplex at neutral pH.

Overall, NMR experiments confirm MD, MD/TI, and CD results, and they demonstrate that the parallel-stranded duplexes studied here are stable and show a Hoogsteen-type hydrogen-bonding pattern similar to that of DNA triplexes. The reverse Watson-Crick model of the parallel-stranded duplex, or the standard antiparallel duplex, is ruled out.

Very extended molecular dynamics simulations fail to provide stable helical structures for sequences containing a similar number of d(A·T) and d(G·C) pairs arranged in the reverse Watson-Crick structure. On the contrary, stable trajectories are found if a Hoogsteen pairing is assumed. The structures obtained in these trajectories allowed us to describe the structure of an H-type parallel duplex, whose overall conformation is close to that displayed by the Hoogsteen strands of a DNA triplex. CD spectra support this and this also agrees with preliminary NMR experiments.

8-aminopurine derivatives are able to largely increase the stability of DNA hairpins containing almost the same number of d(A·T) and d(G·C) duplexes, which are designed to have a parallel arrangement. This increase in stability is accurately represented by state of the art MD and MD/TI calculations when a Hoogsteen-type secondary structure is assumed for the hairpins.

It will be appreciated by those skilled in the art that the present invention provides a new method for the stabilization of parallel-stranded H-type duplexes. The introduction of at least one 8-aminopurine derivative makes stable H duplexes under pH or temperature conditions where the helices will be otherwise unstable. These structures act as templates for the formation of DNA-DNA-DNA and DNA-RNA-DNA triplexes in physiological conditions, which is helpful for biotechnological purposes, as well as for antigene and antisense therapies.

### EXAMPLES

All-Purine Hairpins

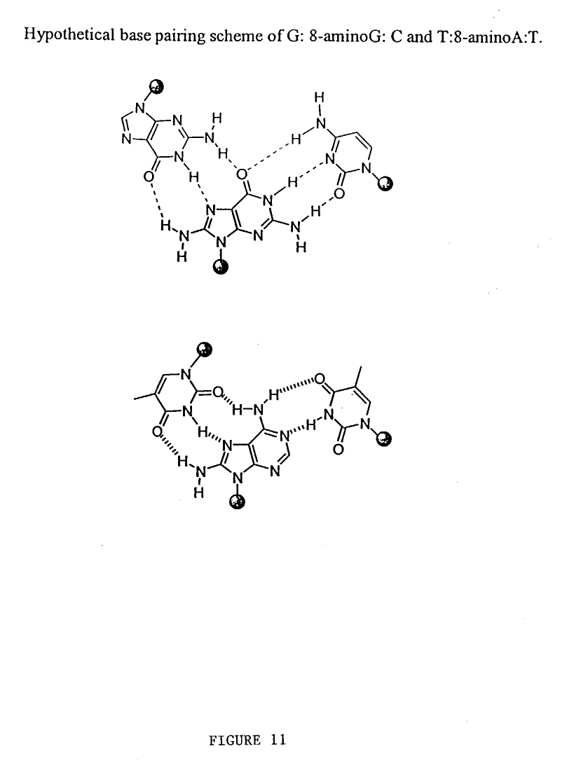

In addition to triplexes having purine:pyrimidine:pyrimidine (type I) triads, it is possible to observe Purine:Pyrimidine:Purine (type II) triads. By observation of the structure of the type II triads, it is possible to draw an extra hydrogen bond between 8-aminopurine (Watson-Crick) and 6-keto of guanine (Hoogsteen) (FIG. 11). Also in the so-called G-T motif it is possible to draw an extra hydrogen bond between 8-aminoadenine and 2-keto of thymine (Hoogsteen). In this way, 8-aminopurine shall stabilize Purine: Pyrimidine: Purine (type II) triplex if Hoogsteen strand is formed by G and A and both 8-aminopurine and 8-aminoadenine may stabilize type TT triplex if Hoogsteen strand is formed by G and T. In both cases 8-aminopurine shall occupy the Watson-Crick purine position. The stability of Type II triplexes is independent of pH. For these reasons they are generally used for triplex applications at physiological pH.

The following oligonucleotides were prepared:

| | | |
|---|---|---|
| h₂₆: | 5′GAAGGAGGAGA-TTTT-TCTCCTCCTTC 3′ | (SEQ ID NO:11) |
| h₂₆-3AG: | 5′GAAGG^{N}AGG^{N}AG^{N}A-TTTT-TCTCCTCCTTC 3′ | (SEQ ID NO:12) |
| h₂₆-3AA: | 5′ GAA^{N}GGA^{N}GGA^{N}GA-TTTT-TCTCCTCCTTC 3′ | (SEQ ID NO:13) |
| s₁₁-GA: | 5′ AGAGGAGGAAG 3′ | (SEQ ID NO:14) |
| s₁₁-GT: | 5′ TGTGGTGGTTG 3′ | (SEQ ID NO:15) |
| RE-2AG: | 5′ GAAGG^{N}AGG^{N}AGA-(EG)₆-AGAGGAGGAAG 3′ | (SEQ ID NO:4, |
| | | SEQ ID NO:14) |
| WC:-11 mer: | 5′TCTCCTCCTTC 3′ | (SEQ ID NO:16) |

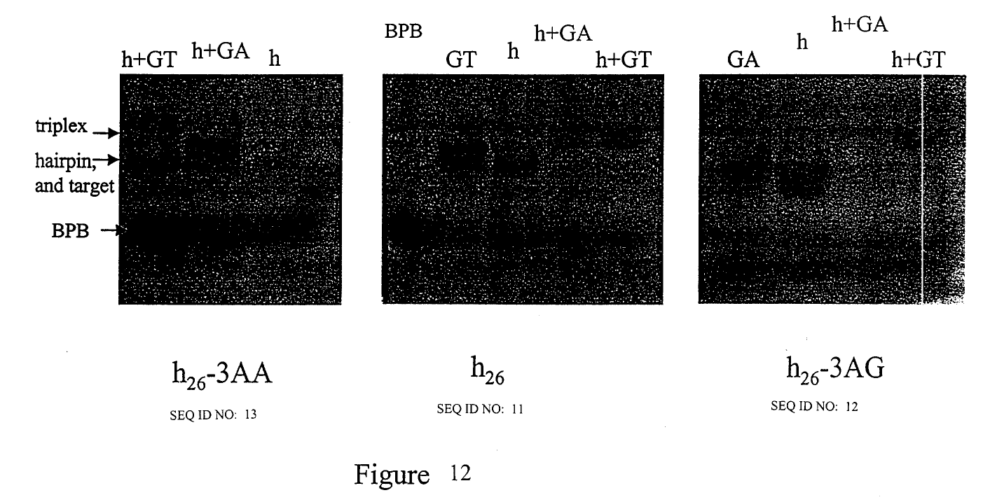

Oligonucleotides s₁₁-GA (SEQ ID NO: 14) and s₁₁-GT (SEQ ID NO: 15)were mixed with h₂₆ derivatives (h₂₆, (SEQ ID NO: 1), h₂₆-3AG (SEQ ID NO: 12) and h₂₆-3AA (SEQ ID NO: 13) in 10 mM sodium cacodilate, 50 mM magnesium chloride and 0.1 mM EDTA pH 7.3. The resulting mixtures were annealed and analyzed on (15%) polyacrylamide gel electrophoresis under native conditions (90 mM Tris-Borate, 50 mM MgCl₂, pH 8.0). The presence of triplex was monitored by the appearance of a slower band (FIG. 12). Unmodified hairpin (SEQ ID NO: 11) and hairpin carrying 8-aminoguanines (SEQ ID NO: 12) gave triplex with both GA- and GT- Hoogsteen oligonucleotides (s₁₁-GA (SEQ ID NO: 14) and s₁₁-GT (SEQ ID NO: 15)). Hairpin carrying 8-aminoadenines (SEQ ID NO: 13) gave only triplex with s₁₁-GT (SEQ ID NO: 15). No triplex was observed with s₁₁-GA (SEQ ID NO: 14) as expected. Melting experiments were also performed in 10 mM sodium cacodilate, 50 mM magnesium chloride and 0.1 mM EDTA. Two transitions were observed: one at 80-85° C. (hairpin to random coil transition) and the other at around 25-30° C. (triplex dissociation). The triplex to duplex transition had a low hyperchromicity and it was difficult to measure with precision. It is known in the art that triplex of type II is accompanied with little or no changes in absorbance.

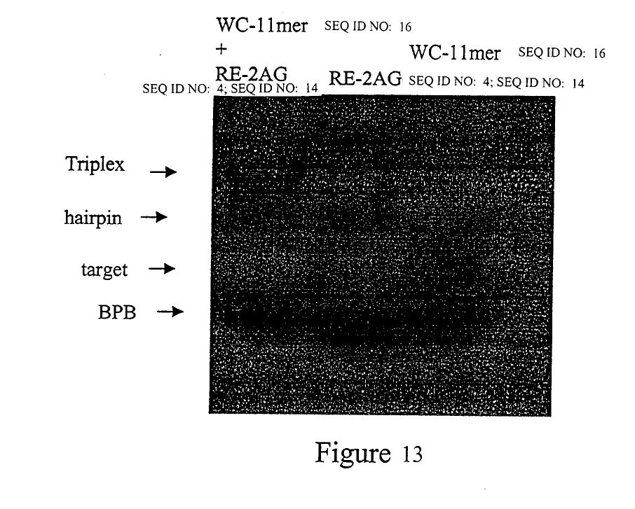

Furthermore, gel-shift analysis was also performed at the same conditions described above with hairpin RE-2AG (SEQ ID NO: 4, SEQ ID NO: 14) and its polypyrimidine target WC-11mer (SEQ ID NO: 16). Also triplex formation was observed by the appearance of a slow moving band (FIG. 13).

Melting experiments of hairpin RE-2AG (SEQ ID NO: 4, SEQ ID NO: 14) alone and hairpin RE-2AG (SEQ ID NO: 4, SEQ ID NO: 14) +WC-11mer (SEQ ID NO: 16) were also performed at 0.1 M sodium phosphate pH 7.2. In these conditions a clear transition (triplex to random coil) was observed with a melting temperature Tm =42° C. Hairpin alone did not show any transition. As a control experiment duplex without the Hoogsteen part (5′ GAAGG^{N}AGG^{N}AGA3′: 3′CTTCCTCCTCT 5′) (SEQ ID NO: 4, SEQ ID NO: 16)showed a melting temperature of 31° C.

Moreover, the triplex stabilization properties of 8-aminopurine were analyzed using the model system described by Pilch et al. (Pilch, D. S., Levenson, C., Schafer, R. H. (1991) Biochemistry, Vol. 30, pages 6081-6087), incorporated by reference herein. Triplexes formed by d(C₃T₄C₃).2[d(G₃A₄G₃)] (SEQ ID NO: 17, SEQ ID NO: 18) and d(C₃T₄C₃).2[d(GG^{N}G^{N}A₄G^{N}G^{N}G)] (SEQ ID NO: 17, SEQ ID NO: 19) were analyzed by melting experiments. Results are shown in Table 5. The substitution of four guanines for 8-aminoguanines changes ΔG of duplex to random coil transition from −12.6 kcal/mol to −10 kcal/mol (a decrease of 2.6 Kcal/mol). On the contrary, the same substitution changes ΔG of triplex to random coil transition from −26.3 kcal/mol to −28.4 kcal/mol (an increase of 2.1 Kcal/mol). We conclude that 8-aminopurine destabilizes duplex but stabilizes type II triplex.

TABLE 5
Thermodynamic parameters of the triplex and the duplex. Data obtained in
10 mM sodium cacodylate, 50 mM MgCl₂ and 0.1 mM EDTA at pH 7.3.

| | ΔH | ΔS | ΔG₂₅ |
|---|---|---|---|
| Structure | (Kcal/Mol) | (cal/mol. ° K.) | (Kcal/mol) |
| natural duplex¹ | −71.6 | −198 | −12.6 |
| 8-aminoG duplex² | −28 | −63 | −10.0 |
| natural triplex¹ | −151.5 | −424 | −26.0 |
| 8-aminoG triplex² | −133 | −350 | −28.4 |

¹natural duplex: d(C₃T₄C₃) · d(G₃A₄G₃) (SEQ ID NO: 17, SEQ ID NO: 18); natural triplex d(C₃T₄C₃) · 2[d(G₃A₄G₃)] (SEQ ID NO: 17, SEQ ID NO: 18)
²8-aminoG duplex: d(C₃T₄C₃). d(GG^{N}G^{N}A₄G^{N}G^{N}G) (SEQ ID NO: 17, SEQ ID NO: 19); 8-aminoG triplex d(C₃T₄C₃) · 2[d(GG^{N}G^{N}A₄G^{N}G^{N}G)] (SEQ ID NO: 17, SEQ ID NO: 19)
ΔG₂₅ refers to the standard free energy change at 25° C.

We conclude that 8-aminopurine stabilizes purine: pyrimidine: purine triplex. These triplexes are formed at physiological pH. 8-Aminoadenine stabilizes type II triplexes if Hoogsteen strand is made out of G and T bases. Hairpins carrying 8-aminoguanines bind polypyrimidine targets by triplex formation and triplexes are stable at physiological pH.

Preparation of Hairpins Containing 8-Aminopurines

Oligonucleotides were prepared on an automatic Applied Biosystems 392 DNA synthesizer. The parallel-stranded hairpins were prepared using methods known by those skilled in the art. 5′-5′ Hairpins (R-22 derivatives) were prepared in three steps. First, the pyrimidine part was prepared using reversed C and T phosphoramidites and reversed C-support (linked to the support through the 5′ end). Second, a linker, such as for example but not limited to, a hexaethyleneglycol linker, was added using a commercially available phosphoramidite. Third, the purine part carrying the modified 8-aminopurines was assembled using standard phosphoramidites for the natural bases and the 8-aminopurine phosphoramidites. The phosphoramidites of 8-aminoadenine, 8-aminopurine and 8-aminohypoxanthine were prepared using methods known by those skilled in the art. For the preparation of 3′-3′ hairpins (B-22 derivatives), a similar approach was used. In this case, the purine part was assembled first, followed by the hexaethyleneglycol. The pyrimidine part was the last to be assembled using reversed phosphoramidites. The phosphoramidite of protected 8-amino-2′-deoxyinosine was dissolved in dry dichloromethane to yield a 0.1 M solution. The remaining phosphoramidites were dissolved in dry acetonitrile (0.1 M solution). Oligonucleotides containing natural bases were prepared using commercially available chemicals and following standard protocols. After the assembly of the sequences, oligonucleotide-supports were treated with 32% aqueous ammonia at 55° C. for 16 h (hour) except for oligonucleotides bearing 8-aminopurine. In this case, a 0.1 M 2-mercaptoethanol solution in 32% aqueous ammonia was used and the treatment was extended to 24 h at 55° C. Ammonia solutions were concentrated to dryness and the products were purified by reversed-phase HPLC. Oligonucleotides were synthesized on a 0.2 μmol scale and with the last DMT group at the 5′ end (DMT on protocol) to facilitate reversed-phase purification. All purified products presented a major peak, which was collected. Yields (OD units at 260 nm after HPLC purification, 0.2 μmol) were between 5-10 OD. HPLC conditions: HPLC solutions were as follows. Solvent A: 5% ACN in 100 mM triethylammonium acetate pH 6.5 solvent B: 70% ACN in 100 mM triethylammonium acetate pH 6.5. Columns: PRP-1 (Hamilton), 250×10 mm. Flow rate: 3ml/min. A 30 min linear gradient from 10-80% B (DMT on) or a 30 min linear gradient from 0-50% B (DMT off).

Binding of Hairpins to Target Sequences by Melting Experiments.

Melting experiments with triple helices were performed as follows. Solutions of equimolar amounts of hairpins and the target Watson-Crick pyrimidine strand (11-mer) were mixed in 0.1 M sodium phosphate/citric acid buffer of pH ranging from 5.5 to 7.0 with or without NaCl or MgCl₂. The DNA concentration was determined by UV absorbance measurements (260 nm) at 90° C., using for the DNA coil state the following extinction coefficients: 7500, 8500, 12500, 12500, 15000 and, 15000 M⁻¹ cm⁻¹ for C, T, G, 8-amino-G, A and, 8-amino-A, respectively. The solutions were heated to about 90° C., allowed to cool slowly to room temperature, and stored at about 4° C. until UV was measured. UV absorption spectra and melting experiments (absorbance vs temperature) were recorded in 1 cm path-length cells using a spectrophotometer, with a temperature controller and a programmed temperature increase rate of 0.5° C. /min. Melts were run on duplex concentration of 4 μM at 260 nm. The samples used for the thermodynamic studies were prepared in a similar way, but melting experiments were recorded at 260 nm and using 0.1, 0.5 and 1 cm path-length cells.

Thermodynamic data were analyzed using methods known by those skilled in the art. Melting curves were obtained at concentrations ranging from 0.5 to 25 μM of triplex. The melting temperatures Tm were measured at the maximum of the first derivative of the melting curve. The plot of 1/Tm versus InC was linear. Linear regression of the data gave the slope and the y-intercept, from which ΔH, and ΔS were obtained. The free energy was obtained from the standard equation: ΔG=ΔH-TΔS.

Binding of Hairpins to Target Sequences by Gel-Shift Experiments.

The binding of hairpins to their polypyrimidine targets was analyzed by gel retardation assays. The following targets were studied: WC-11 mer: ^{5′}TCT CCT CCT TC^{3′}(SEQ ID NO: 16) and T31-PYR: 5′ CGA GTC ATT GTC TCC TCC TTC AGT CAT CGA G 3′. (SEQ ID NO: 20).

Either the target oligonucleotides or the hairpins were radioactively labeled at the 5′ end by T4 polynucleotide kinase and [γ-³²P]-ATP with 35-50 μmol of the oligonucleotide dissolved in 20 μl of kinase buffer. After incubation at 37° C. for 45 min (minutes), the solution was heated to 70 C. for 10 min to denature the enzyme and the solution was cooled to room temperature. 60 μl of 50 mM potassium acetate in ethanol was added to the solution and the mixture was left at −20° C. for at least 3 h. The mixture was centrifuged at 4° C. for 45 min (14000 rpm) and the supernatant was removed. The pellet was washed with 60 μl of 80% ethanol and centrifuged for 20 min at 4° C. The supernatant was removed and the pellet was dissolved in 0.2 ml of water.

The radiolabelled target was incubated with the hairpins in 0.1 M sodium phosphate/citric acid buffer of pH ranging from 5.5 to 7.0 at room temperature for 30-60 min. The hairpins were added in increasing amounts from 2 to 200 molar equivalents. After incubation, the mixtures were analysed by 15% polyacrylamide gel electrophoresis at room temperature using the same buffer as for the incubation: 0.1 M sodium phosphate/citric acid buffer of pH ranging from 5.5 to 7.0. The formation of the triplex was monitored by the appearance of a radioactive band with less mobility than the band corresponding to the target alone.

Experiments carried out with radiolabelled hairpins were performed in a similar way. In this case, increasing amounts from 2 to 200 molar equivalents of target oligonucleotide were added to the hairpin.

Circular Dichroism

Oligonucleotides were dissolved in 100 mM phosphate buffer pH 6.0, 50 mM sodium chloride and 10 mM magnesium chloride. The equimolar concentration of each strand was 4-5 μM. The solutions were heated to about 90° C., allowed to cool slowly to room temperature and stored at about 4° C. until CD was measured. The CD spectra were recorded on a Jasco J-720 spectropolarimeter attached to a Neslab RP- 100 circulating water bath in 1 cm path-length quartz cylindrical cells. Spectra were recorded at room temperature using a 10 nm/min scan speed, a spectral band width of 1 nm and a time constant of 4 s. CD melting curves were recorded at 280 nm using a heating rate of 20° C./h and a scan speed of 100 nm/min. Al the spectra were subtracted with the buffer blank, normalized to facilitate comparisons and noise-reduced using Microcal Origin 5.0 software.

NMR Spectroscopy

An equimolar mixture of hairpin d(3′-AG A^{N} GG A^{N} GGA AG-5′-(EG)₆-5′-CTT CCT CCT CT-3′) (A^{N}=8-amino-A) (SEQ ID NO: 3, SEQ ID NO: 2) and WC-11mer: ^{5′}TCT CCT CCT TC^{3′} (SEQ ID NO: -16) was prepared in 250 μl of 9:1 H₂O/D₂O, 25mM sodium phosphate buffer and 100 mM NaCl. The pH was adjusted by adding small amounts of concentrated HCl. The final oligonucleotide concentration was around 1 mM. Spectra were acquired in a Bruker AMX spectrometer operating at 600 MHz and processed with the UXNMR software. Water suppression was performed using a jump-and-return pulse sequence with null excitation in the water signal. All experiments were carried out at 5° C.

Molecular Modeling

Two types of calculations were made to test whether parallel-stranded hairpins behave as a template for triplex formation: i) quantum mechanics, and ii) classical molecular dynamics.

Quantum Mechanical Calculations.

The energy of the Watson-Crick hydrogen bonding of adenine (or 8-aminoadenine) and thymine, and guanine (or 8-aminopurine) and cytosine was computed at the B3LYP/6-31G(d) level for the isolated purines, and for the preformed Hoogsteen dimer adenine (or 8-aminoadenine)-thymine or guanine (or 8-aminopurine)-cytosine⁺ (FIG. 14). The geometries of monomers (A, A^{N}, G, G^{N}, C, T and C⁺), dimmers (A-T, A.T, A^{N}-T, A^{N}.T, G-C^{+, l G}^{N}-C⁺, G.C and G^{N} C), and trimers (T-A.T, T-A^{N}.T, C⁺-G.C, and C⁺-G^{N}.C) were fully optimized at the B3LYP/6-31G(d) level of theory (Watson-Crick base pair is indicated with a dot, Hoogsteen base pair is indicated with a dash). Optimized geometries were subjected to frequency analysis. Basis-set superposition errors (BSSE) were corrected following Boys & Bernardi.

Molecular Dynamics.

Trajectrories for poly d(T-A.T), poly d(T-A) and poly(A.T) were obtained by classical molecular dynamics. Starting structures for our simulations were surrounded by cations to achieve neutrality, hydrated (around 2-3 thousand molecules), optimized, thermalized and equilibrated following standard multistage protocol as known by those skilled in the art. Simulations were carried out for 1.5 ns at constant pressure and temperature (P=1 atm., T=298° K.) in periodic boundary conditions using the particle mesh Ewald technique (PBC-PME). Only the last 1 ns of the trajectories were considered for the analysis. SHAKE was used to constrain all the bonds at optimum lengths, which allowed us to use a 2 fs. time step for integration of Newton's laws. TIP3P and AMBER-98 force-field, supplemented with specific parameters for protonated cytosine and 8-aminopurines were used to describe molecular interactions. Quantum mechanical calculations were made using the Gaussian-94 computer program. Molecular dynamic simulations were performed using the AMBER-95 suite of programs.

Structure of the Oligonucleotide Derivatives.

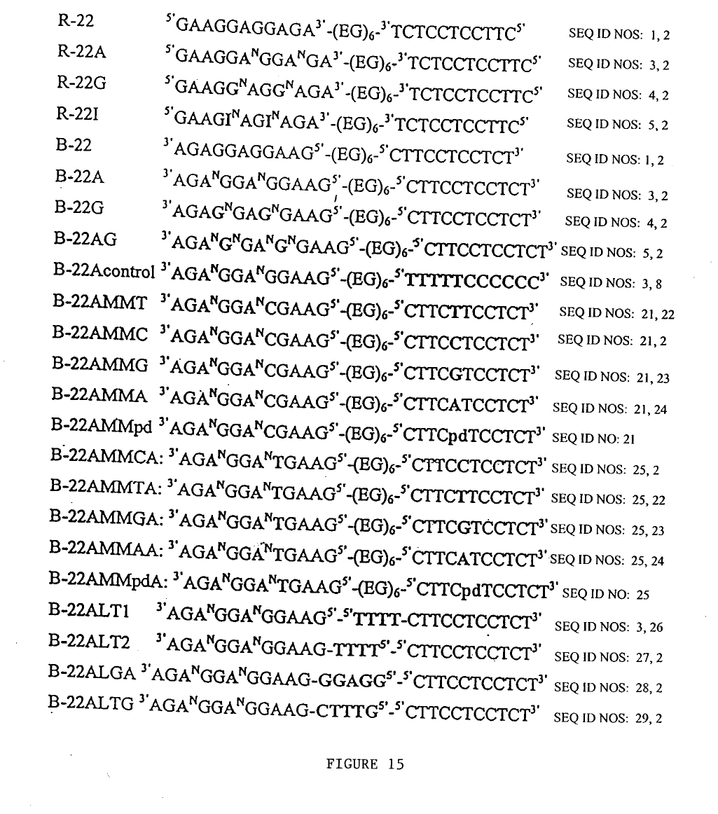

The binding properties of hairpins carrying 8-aminoadenine (A^{N}), 8-aminopurine (G^{N}) and 8-aminohypoxanthine (I^{N}) connected head-to-head to the Hoogsteen pyrimidine strand were studied. The sequences of the oligonucleotides are shown in FIG. 15. The target DNA sequence comprises a triplex characterized by Xodo et al.. Here, the polypyrimidine Hoogsteen strand was linked to the Watson-Crick polypurine strand.

The first group of hairpins (R-22 (SEQ ID NO: 1, SEQ ID NO: 2) R-22A (SEQ ID NO: 3, SEQ ID NO: 2) R-22G (SEQ ID NO: 4, SEQ ID NO: 2) R-22I (SEQ ID NO: 5, SEQ ID NO: 2) are parallel-stranded and connected through their 3′ ends with a hexaethyleneglycol linker [(EG)₆]. They contain 22 bases and two purines replaced by the corresponding 8-aminopurines. In hairpin R-22A (SEQ ID NO: 3, SEQ ID NO: 2) two adenines are replaced by two 8-aminoadenines (A^{N}); in hairpin R-22G (SEQ ID NO: 4, SEQ ID NO: 2)two guanines are replaced by two 8-aminoguanines (G^{N}) and in hairpin R-22I (SEQ ID NO: 5, SEQ ID NO: 2), two guanines are substituted by two 8-aminohypoxanthines (I^{N}). Hairpin R-22 (SEQ ID NO: 1, SEQ ID NO: 2) is a control sequence that contains only the natural bases without modification. The number of modified bases in each hairpin was selected to optimize stability with a minimum number of modified bases, as described elsewhere.

The second group of hairpins B-22 (SEQ ID NO: 1, SEQ ID NO: 2) B-22A (SEQ ID NO: 3, SEQ ID NO: 2) B-22G (SEQ ID NO: 4, SEQ ID NO: 2) have a similar composition but the polypurine and the polypyrimidine parts are connected through their 5′ ends with a hexaethyleneglycol linker [(EG)₆]. In addition, a hairpin bearing two 8-aminoguanines and two 8-aminoadenines was prepared (B-22AG (SEQ ID NO: 34, SEQ ID NO: 2) to test whether the stabilizing properties of the two 8-aminopurines are additive. A control oligonucleotide (B-22A control (SEQ ID NO: 3, SEQ ID NO: 8)) with the same sequence in the polypurine part as B-22A (SEQ ID NO: 3, SEQ ID NO: 2) but a random polypyrimidine sequence was prepared. Finally, the oligomers B-22AMMT (SEQ ID NO: 21, SEQ ID NO: 22), B-22AMMC (SEQ ID NO: 21, SEQ ID NO: 2), B-22AMMG (SEQ ID NO: 21, SEQ ID NO: 23), B-22AMMA (SEQ ID NO: 21, SEQ ID NO: 24), B-22AMMpd (SEQ ID NO: 21), B-22AMMCA (SEQ ID NO: 25, SEQ ID NO: 2), B-22AMMTA (SEQ ID NO: 25, SEQ ID NO: 22), B-22AMMGA (SEQ ID NO: 25, SEQ ID NO: 23), B-22AMMAA (SEQ ID NO: 25, SEQ ID NO: 24) and B-22AMMpdA (SEQ ID NO: 25) were prepared to study the effect of an interruption on the stability of the triple helix. In these hairpins, two adenines are replaced by two 8-aminoadenines. A pyrimidine (C or T) is located in the middle position of the purine part, and each of the natural bases and an abasic site model compound (propanediol, pd) are located in the corresponding position at the Hoogsteen strand.

A third group of oligomers (B-22ALT1 (SEQ ID NO: 3, SEQ ID NO: 26), B-22ALT2 (SEQ ID NO: 27, SEQ ID NO: 2), B-22ALGA (SEQ ID NO: 28, SEQ ID NO: 2), B-22ALTG (SEQ ID NO: 29, SEQ ID NO: 2) and B-22N) have the same nucleotide sequence as B-22A but the loop between the polypurine and polypyrimidine parts is made out of nucleotides (-TTTT-, -GGAGG-, -CTTTG-) instead of the hexaethyleneglycol bridge.

Thermal Stability of the Triplex Formed by Hairpins Linked by 3′-3′ Bonds.

The relative stability of triple helices formed by R-22 hairpin derivatives and the polypyrimidine target sequence (WC-11mer (SEQ ID NO: 16)) was measured spectrophotometrically at various pHs (pH 4.5-7.0). In almost all cases, one single transition was observed with a hyperchromicity around 25% at acidic pH and 20% at neutral pH. The melting curve was assigned to the transition from triple helix to random coil. Exceptionally, the melting curve of the triplex R-221: WC-11mer (SEQ ID NO: 5, SEQ ID NO: 2, SEQ ID NO: 16) at about pH 5.5 and 6 showed two pH-dependent transitions. When A and G were replaced by 8-aminoadenine (A^{N}) and 8-aminopurine (G^{N}) in the triple helix, this was greatly stabilized (10-18° C. in the range from about pH 4.5 to pH 7.0, Table 6). When guanine was replaced by 8-aminohypoxanthine (I^{N}) triple helix stability increased only slightly at acidic pH, but the triplex containing I^{N} maintained its stability at neutral pH while the unmodified triplex stability rapidly decreased.

To test whether transition was due to triple helix formation, melting curves were obtained with hairpins (R-22 (SEQ ID NO: 1, SEQ ID NO: 2) R-22A (SEQ ID NO: 3, SEQ ID NO: 2) R-22G (SEQ ID NO: 4, SEQ ID NO: 2) R-22G (SEQ ID NO: 5, SEQ ID NO: 2) alone, in the absence of the polypyrimidine target sequence (WC-11 mer) (SEQ ID NO: 16). A single transition was also observed but at lower temperature and with a hyperchromicity around 10-15%, indicating that the transition observed with the WC-11mer (SEQ ID NO: 16) (triple helix) differs from that observed without the WC-11mer (SEQ ID NO: 16). The transition observed in the hairpins alone corresponds to the parallel duplex to random coil transition.

Hairpins Linked by 3′-3′ Bonds Versus Hairpins with 5′-5′ Linkages

In addition to the hairpins linked by 3′-3′ bonds (R-22 derivatives), hairpins linked by 5′-5′ bonds (B-22 derivatives) were prepared. The relative stability of triple helices formed by the B-22 oligonucleotide derivatives and the polypyrimidine target sequence (WC-11mer) (SEQ ID NO: 16) was measured. As described herein, one single transition was observed with a hyperchromicity around 25%, which was assigned to the melting of the triple helix. Replacement of A by 8-aminoadenine (A^{N}) and guanine by 8-aminopurine (G^{N}) in triple helix greatly stabilized the triple helix (Table 7). Moreover, when the melting curves of the hairpins were analyzed without the target WC-11mer (SEQ ID NO: 16), the parallel structure was stabilized by the presence of 8-aminopurines. At acidic pH triplexes formed by both types of hairpins have similar stability. At neutral pH hairpins linked by 3′-3′ bonds (R-22 derivatives) form more stable triplexes than hairpins linked by 5′-5′ bonds (B-22 derivatives). Nevertheless, the increase in stability due to 8-aminopurines was similar in both systems.

Next, we examined whether the stabilization properties of 8-aminoadenine and 8-aminopurine are additive. A hairpin with two 8-aminoadenines and two 8-aminoguanines substitutions was prepared (B22AG) (SEQ ID NO: 34, SEQ ID NO: 2). Melting curves were obtained with the appropriate hairpin and the target WC-11mer at pHs between 4.5-7.0, 0.1 M sodium phosphate, citric acid, 1 M NaCl. We found that the stabilization properties of the 8-aminopurines are additive (Table 7). For example, at pH 6.0 the addition of the two 8-aminoguanines and two 8-aminoadenines raises the melting temperature by 20° C., whereas two 8-aminoadenines induce an increase of 6° C., and two 8-aminoguanines a rise of 14° C.

Role of the Hoogsteen Strand on the Triplex Formation of Hairpins

The role of the Hoogsteen strand was further investigated. We prepared a hairpin probe of the same purine sequence, but with two 8-aminoadenine substitutions and a non-complementary pyrimidine strand. This oligonucleotide (named B-22A control (SEQ ID NO: 3, SEQ ID NO: 8)) can only form Watson-Crick interactions with the target sequence (WC-11mer (SEQ ID NO: 16)).

When the Hoogsteen strand is replaced by a non-complementary sequence, the structure of the parallel duplex is lost, as revealed by the disappearance of the transition observed when the melting curve is obtained without the target WC-11mer (SEQ ID NO: 16) (Table 8).

The transitions observed with the duplexes formed by B22-A control: WC-11mer (SEQ ID NO: 3, SEQ ID NO: 8, SEQ ID NO: 16) showed lower Tm and hyperchromicity. The hyperchromicity associated with the transition of the duplex formed by B-22A control: WC-11mer (SEQ ID NO: 3, SEQ ID NO: 8, SEQ ID NO: 16) was 11%, which is indicative of a duplex-to-single-strand transition.

The transitions observed with the complex formed by B-22A: WC-11mer (SEQ ID NO: 3, SEQ ID NO: 2, SEQ ID NO: 16) showed a 22% hyperchromicity, indicating a triplex-to-single-strand transition. The difference between the Tm of the B-22A control duplex and B-22A triplex is the gain obtained by the addition of the Hoogsteen strand. At about pH 6.0, this difference is of 11° C. (1.0° C. per base) and at pH 5.5, it is of 16° C. (1.4° C. per base).

Salts Effects on Triplex Stability.

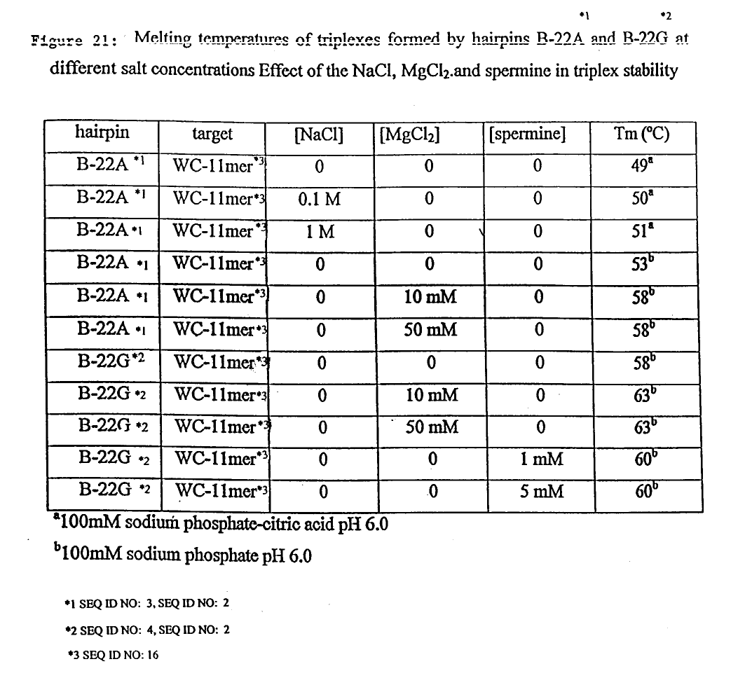

Next, we studied the effect of NaCl Mg C12 and spermine on the stability of triplexes at pH 6.0. (FIG. 21). We used the triplex formed by the hairpin B-22A (SEQ ID NO: 3, SEQ ID NO: 2) and the WC-11mer (SEQ ID NO: 16), as well as the triplex formed by the hairpin B-22G (SEQ ID NO: 4, SEQ ID NO: 2) and WC-11mer (SEQ ID NO: 16). For NaCl, the buffer used was 0.1 M sodium phosphate-citric acid pH 6.0. For MgCl₂ and spermine, the buffer used was 0.1 M sodium phosphate pH 6.0.

Sodium chloride had a slight stabilization effect (from about 49° C. (without NaCl) to 51° C. (1 M NaCl)). Low concentrations of MgCl₂ stabilize the triplex, e.g. the melting temperature of triplexes B-22G: WC-11mer (SEQ ID NO: 4, SEQ ID NO: 2, SEQ ID NO: 16) and B-22A: WC-11mer increased by 5 degrees from no MgCl₂ to 10 mM MgCl₂. From 10 mM to 50 mM MgCl₂, the increase in melting temperature is nil or lower than one degree. In a preferred embodiment of this invention the presence of magnesium is employed for enhancing the stability of the triplex, including wherein the concentration of magnesium is more preferably 10 mM. Spermine does not generally affect the stability of triplexes.

Presence of Interruptions in the Polypyrimidine Target Sequence.

We also assessed the effect of an interruption on the polypyrimidine track of the target. To this end, two polypyrimidine targets with a purine in the middle of the sequence were prepared (s₁₁-MMG: ^{5′}TCT CCT GCT TC^{3′} (SEQ ID NO: 30) and s₁₁-MMA: ^{5′}TCT CCT ACT TC^{3′}) (SEQ ID NO: 31). Next, hairpins carrying the four natural bases and an abasic model compound, such as for example but not limited to pd (propanediol), at the Hoogsteen position were prepared (FIG. 15). Moreover, two 8-aminoadenines were introduced in the purine part. These oligomers have the complementary base at the Watson-Crick position opposite to the interruption and a T, C, G, A or pd on the Hoogsteen strand opposite to the interruption. Melting curves were obtained at pH 6.0, 0.1 M sodium phosphate, 1 M NaCl.

The melting temperatures of triplexes carrying a guanine on the polypyrimidine target instead of a cytosine are shown in Table 9. The hairpin with a cytosine in the Hoogsteen pyrimidine part gave the best binding. Hairpin B-22AMMC (SEQ ID NO: 21, SEQ ID NO: 2) bound to its target (s₁₁-MMG) (SEQ ID NO: 30), although the Tm decreased by 4° C. (47° C. B-22AMMC: s₁₁-MMG (SEQ ID NO: 21, SEQ ID NO: 2, SEQ ID NO: 30) compared with 51° C. B-22A: WC-11mer (SEQ ID NO: 3, SEQ ID NO: 2, SEQ ID NO: 16). The binding of the new hairpin to its new target is very selective as revealed by the marked decrease in the Tm of the triplex B-22AMMC (SEQ ID NO: 21, SEQ ID NO: 2) with the old target (33° C. B-22AMMC: WC-11mer (SEQ ID NO: 21, SEQ ID NO: 2, SEQ ID NO: 16) versus 47° C. B-22AMMC: s₁₁-MMG (SEQ ID NO: 21, SEQ ID NO: 2, SEQ ID NO: 30).

A similar result was obtained when an adenine was introduced in the polypyrimidine target (Table 10). In this case, the best base at the Hoogsteen position was G. The preference of G to bind to A.T interruptions and the preference of C to bind G.C interruptions are well known by those skilled in the art. However, the interruptions in parallel hairpins are easier to overcome because of the purine Watson-Crick part. Thus parallel hairpins, especially hairpins carrying 8-aminopurines, can be redesigned to bind efficiently to polypyrimidine targets carrying a short interruption.

Role of the Loop on Triplex Stability.

Finally, the role of the loop on the stability of the triplex was analysed by preparing derivatives of B-22A with various loops. In addition to the hexaethyleneglycol linker, the nucleotide loops -TTTT-, -GGAGG-, and -CTTTG- were studied. Two tetrathymine loops were prepared: one oppositely oriented to the purine strand (B-22ALT10 (SEQ ID NO: 3, SEQ ID NO: 26) and the second in the same orientation as the purine strand (B-22ALT2 (SEQ ID NO: 27, SEQ ID NO: 2)). The GGAGG and CTTTG loops were in the same orientation as the purine strand (B-22ALGA (SEQ ID NO: 28, SEQ ID NO: 2) and B-22ALTG (SEQ ID NO: 29, SEQ ID NO: 2). The melting curves of triplexes formed by hairpins and target WC-11mer (SEQ ID NO: 16) were obtained at pH 6.0, 0.1 M sodium phosphate, 1 M NaCl. Melting temperatures are set forth below.

| | | | |
|---|---|---|---|
| B-22A | ^{3′}AGA^{N} GGA^{N} GGAAG^{5′}-(EG)₆₋^{5′-}CTTCCTCCTCT^{3-′} | Tm = 51° C. | (SEQ ID NO:3, SEQ ID NO:2) |
| B-22ALT1 | ^{3′}AGA^{N} GGA^{N} GGAAG^{5′}-^{5′}TTTT-CTTCCTCCTCT^{-3′} | Tm = 57° C. | (SEQ ID NO:3, SEQ ID NO:26) |
| B-22ALT2 | ^{3′}AGA^{N}GGA^{N}GGAAG-TTTT^{-5′}-^{5′}CTTCCTCCTCT^{3′} | Tm = 55° C. | (SEQ ID NO:27, SEQ ID NO:2) |
| B-22ALGA | ^{3′}AGA^{N}GGA^{N}GGAAG-GGAGG^{5′}-^{5′}CTTCCTCCTCT^{3′} | Tm = 54° C. | (SEQ ID NO:28, SEQ ID NO:2) |
| B-22ALTG | ^{3′}AGA^{N}GGA^{N}GGAAG-CTTTG^{5′}-^{5′}CTTCCTCCTCT^{3′} | Tm = 54° C. | (SEQ ID NO:29, SEQ ID NO:2) |

Use of nucleotide loops is more preferable for the stability of the triplex. Best results were obtained with the reversed TTTT linker (hairpin B-22ALT1 (SEQ ID NO: 3, SEQ ID NO: 26), ΔTm 6° C.), followed by the TTTT linker (hairpin B-22ALT2 (SEQ ID NO: 27, SEQ ID NO: 2), ΔTm 4° C.) and the GGAGG and CTTTG linkers (hairpin B-22ALGA (SEQ ID NO:28, SEQ ID NO: 2), B-22ALTG (SEQ ID NO: 29, SEQ ID NO: 2), ΔTm 3° C.). While not wishing to be bound by a particular theory, we suggest that the enhanced stability found with hairpins having nucleotide loops is due to the high salt concentrations used in the melting experiments. When melting experiments were performed at lower salt concentrations, it was found that the differences between hairpins with the hexaethyleneglycol linker and nucleotide linkers were less pronounced.

Molecular Modeling

The formation of a triple helix by the binding of a Hoogsteen parallel-stranded duplex to a single-stranded oligonucleotide is guided by the formation of Watson-Crick-like H-bonds. The presence of the complementary Hoogsteen base may alter the magnitude of the Watson-Crick interaction. Results demonstrate that no dramatic changes can be expected in the Watson-Crick interaction by the presence of the Hoogsteen base. Thus, the binding of T to the Hoogsteen A-T (or A^{N}-T) dimer is less than 1 kcal/mol worse than the binding to A and the binding of C to the protonated Hoogsteen dimer G-C (or G^{N}-C) is 2-3 kcal/mol better than the binding to an isolated G. The presence of 8-aminopurines might slightly decrease in the intensity of Watson-Crick interactions, but without affecting the formation of triplexes from Hoogsteen duplexes, as reported elsewhere.

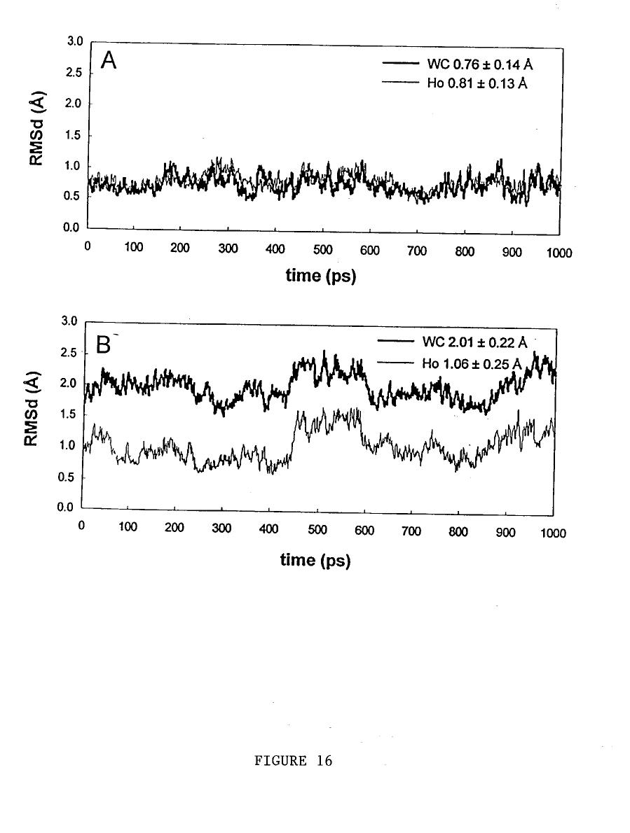

Calculations suggest that a pre-organized Hoogsteen duplex gives rise to a triplex. However, the isolated Hoogsteen duplex may not be sufficiently pre-organized. The magnitude of the pre-organization work can be estimated by the mean root mean square deviation (RMSd) between the structures sampled during the trajectories of the isolated duplex and the average structure of the duplex in the triplex structure. FIG. 16 displays the RMSd between the trajectories of both Watson-Crick and Hoogsteen duplexes and the average structures of both duplexes when incorporated inside the triplex (average structure obtained by analysis of the MD trajectory of the triplex). The RMSd between the free Hoogsteen and the triplex-preorganized Hoogsteen duplex is only around IA, near the thermal noise of the simulation, as revealed by the fact that the RMSd between the trajectories of the isolated duplexes (Hoogsteen or Watson-Crick) and the corresponding MD-averaged structures is about 0.8 Å. In contrast, the RMSd between the free Watson-Crick duplex and the triplex-preorganized Watson-Crick duplex is about 2 Å. MD simulations strongly suggest that the free parallel Hoogsteen duplex is better pre-organized to form a triplex than the Watson-Crick antiparallel duplex. This finding agrees with the CD data, which show that the spectra changes more in the transition from a Watson-Crick duplex to triplex than in the transition from a Hoogsteen duplex to the corresponding triplex. Therefore, it will be appreciated by those skilled in the art that the Hoogsteen parallel hairpins of this invention are very efficient templates for the formation of triple helices.

Thenrodynamic Studies

The dependence of the triplex to random coil transition on DNA concentration was studied on several triplexes (Table 12). In all cases, the melting temperatures of the triplex to random coil transitions decrease with the concentration, as expected for a bimolecular transition. The plot of 1/Tm versus In concentration was linear, giving a slope and a y-intercept from which ΔH, ΔS and ΔG were obtained (Table 12).

The ΔG for the triplex dissociation was −58 kJ/mol for the unmodified triplex, −76 kJ/mol for the triplexes carrying two A^{N} and −88 kJ/mol for the triplex carrying two G^{N}. Comparison between these values gives a difference in ΔG of approximately 17 kJ/mol for two A→A^{N} substitutions (7.5 kJ/mol 2.0 Kcal/mol per substitution). For the triplex carrying G^{N}, the difference in ΔG is 30 kJ/mol (15 kJ/mol, 3.6 Kcal/mol per substitution). Compared with other base analogues, these are among the highest triplex stabilization properties reported for a modified base, although we measured the stability of Hoogsteen and Watson-Crick base pairs jointly.

Gel-Shift Assays

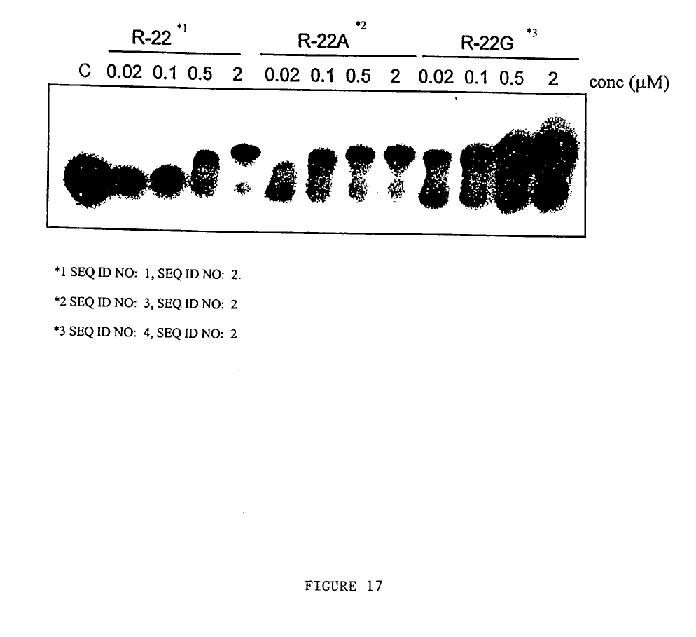

The binding of hairpins to their targets was also analyzed by gel-shift experiments. The target was labeled radioactively with [γ-³²P]-ATP and polynucleotide kinase and increasing amounts of the hairpins were added. After incubation at room temperature from about 30 min-1 hr in a citric-phosphate buffer pH 6 of 100 mM Na⁺ ionic strength, the mixtures were analyzed by polyacrylamide gel electrophoresis. The formation of the triplex was monitored by the appearance of a radioactive band with less mobility than the band corresponding to the target alone (FIG. 17).

FIG. 17 shows the binding of hairpins (SEQ ID NO: 1, SEQ ID NO: 2) R-22A (SEQ ID NO: 3, SEQ ID NO: 2) and R-22G (SEQ ID NO: 4, SEQ ID NO: 2) to the single-stranded target WC-11 mer (^{5′}TCTCCTCCTTC^{3′}) (SEQ ID NO: 16). In all cases, a new radioactive band with lower mobility appeared. The relative intensity of this new band is consistent with the melting experiments. For example, the hairpins carrying the modified purines (A^{N} and G^{N}) completed the formation of the new band at a lower concentration (0.02-0.1 μM, FIG. 17) than the unmodified hairpin (0.5 μM, FIG. 17). The hairpin carrying G^{N} also showed better binding properties than the hairpin carrying A^{N}, in agreement with melting experiments. Moreover, binding is more efficient at pH 5.0 than at pH 7.0. We also found that, the binding of hairpin B-22A control, (SEQ ID NO: 3, SEQ ID: 8) which had a non-functional Hoogsteen strand, with its target WC-11mer (SEQ ID NO: 16) gave a low mobility band, but at concentrations 100 fold higher than hairpin B-22A (SEQ ID NO: 3, SEQ ID NO: 2). All these data indicate that the lower mobility bands detected with hairpins correspond to the triplex.

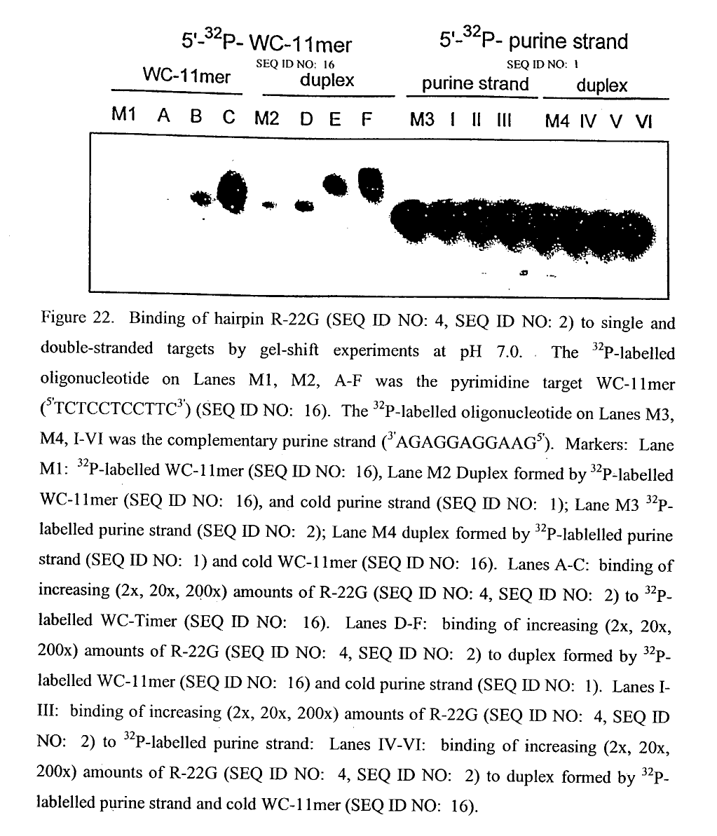

The binding of hairpin R-22G (SEQ ID NO: 4, SEQ ID NO: 2) to the single-stranded target WC-11mer (SEQ ID NO: 16) and a double-stranded target formed by the WC-11mer (^{5′}TCTCCTCCTTC^{3′}) (SEQ ID NO: 16) labeled and its complementary purine strand (^{3′}AGAGGAGGAAG^{5′}) (SEQ ID NO: 1) was also examined. (FIG. 22). When the labeled oligonucleotide is the target pyrimidine strand (WC-11 mer) (SEQ ID NO: 16) a new radioactive band with lower mobility appear in both single and double-stranded targets, revealing the formation of the triplex. In contrast, when the labeled oligonucleotide is the purine strand, no new band is observed, indicating that hairpins only bind to the target pyrimidine strand.

In addition, the binding of hairpins to a second set of single- and double-stranded DNA targets longer than about 11 bases were studied. The double-stranded DNA target had 31 base pairs containing an 11 base pyrimidine track complementary to the hairpins described in this study at the middle of the molecule:

(SEQ ID NO:32)

| | |
|---|---|
| T₃₁ | 5′ CGAGTCATTGTCTCCTCCTTCAGTCATCGAG 3′ |

(SEQ ID NO:33)

| | |
|---|---|
| T₃₁ compl. | 3′ GCTCAGTAACAGAGGAGGAAGTCAGTAGCTC 5′ |

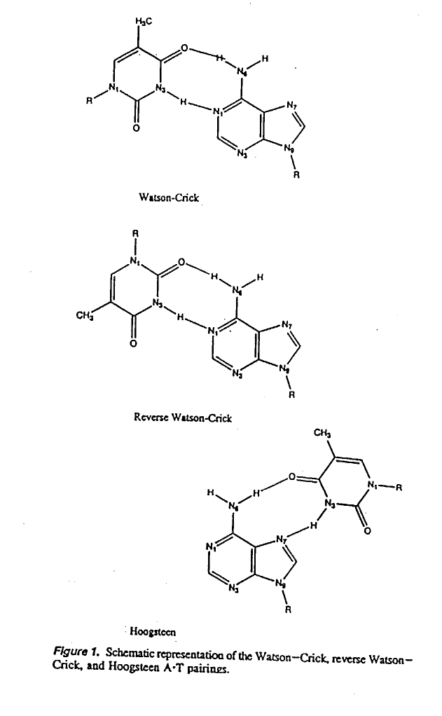

The binding of hairpins to single-stranded targets (T₃₁) (SEQ ID NO: 32) was clearly detected (FIG. 1 ). In contrast, hairpins did not bind to double-stranded DNA. While not wishing to bound by a particular theory, the differences in binding on double-stranded DNA targets may be due to the fact that small duplexes contain a large population of single-stranded molecules in equilibrium with the double-stranded form. The hairpin probably binds the single-stranded form, thus displacing the double-stranded form to the triplex. In longer duplexes, single-stranded forms are scarce, and so the hairpin has to bind and open the duplex to displace the complementary strand. For the hairpins described herein, this phenomenon may be very slow or impracticable.

When the binding experiment was performed by addition of excess of cold target T₃₁ (SEQ ID NO: 32) to radio-labelled hairpin (R22G) (SEQ ID NO: 4, SEQ ID NO: 2), triplex formation was also observed (FIG. 5). Radiolabelled hairpin (R22G) (SEQ ID NO: 4, SEQ ID NO: 2) alone showed two bands in native gels. The fast running band showed the mobility expected for an oligonucleotide of 22 bases. The slow running band had the mobility of a dimer. It is believed that this second band corresponds to the parallel dimer. Thus parallel hairpins are in equilibrium between the intramolecular hairpin and the intermolecular dimer. When the polypyrimidine target was added, the mobility of the dimer varied enough to show complete formation of the band corresponding to the triplex. Formation of the triplex of the parallel dimer was not observed.

Circular Dichroism

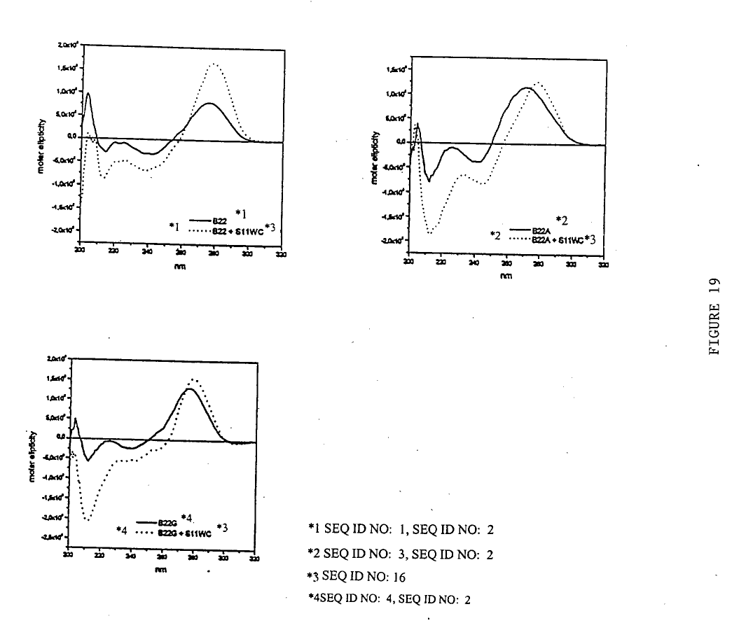

To confirm triplex formation and gain more information on the structure of the hairpins, circular dichroism (CD) spectra were obtained. This technique measures the differences in the absorption of polarized light. Changes in the conformation of nucleic acids can be detected by CD and comparison of the spectra with the spectra of known structures suggests the presence of a particular conformation. The appearance of an intense negative short-wavelength (210-220 nm) band in the CD spectra indicates the formation of a triple-stranded complex. The CD spectra of the triplex formed between the hairpins B-22 (SEQ ID NO: 1, SEQ ID NO: 2) B-22A (SEQ ID NO:3, SEQ ID NO: 2) and B-22G (SEQ ID NO: 4, SEQ ID NO: 2) and their targets as well as the CD spectra of the hairpins alone are shown in FIG. 19. In all cases, we observed an intense negative band (near 215 nm) upon binding of the hairpins with the target molecule. The intensity of the negative band correlates with the strength of the interactions because the negative band is more intense with the triplex formed by modified hairpins (B22-A (SEQ ID NO:3, SEQ ID NO: 2) and B-22G (SEQ ID NO: 4, SEQ ID NO: 2)) which are more stable by melting experiments.

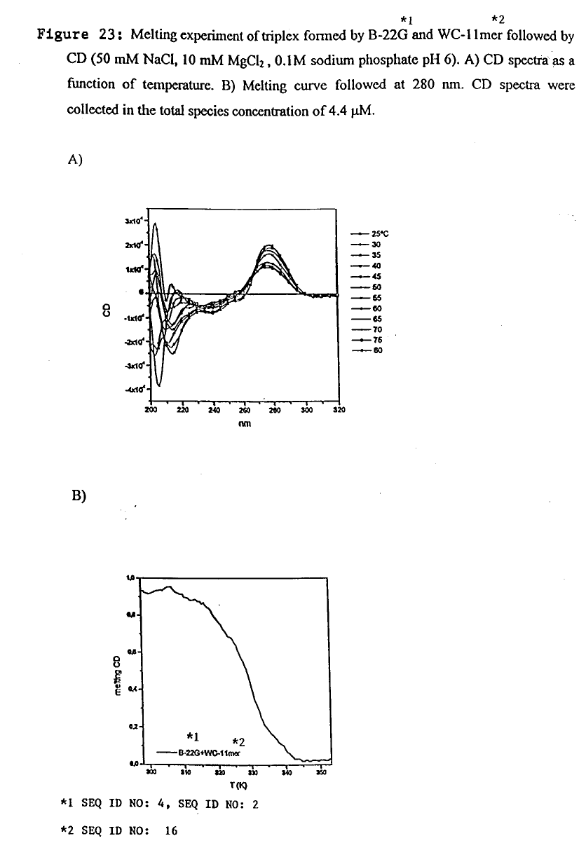

The melting curves of triplexes formed by hairpins B-22A (SEQ ID NO: 3, SEQ ID NO: 2) and B-22G with their polypyrimidine target (WC-11mer (SEQ ID NO: 16)) were also analyzed by CD spectrometry. Melting temperatures obtained by CD experiments were similar to temperatures observed by UV absorption (53.0° C. for triplex B-22A: WC-11mer (SEQ ID NO: 3, SEQ ID NO: 2, SEQ ID NO: 16), 56.5° C. for triplex B-22G: WC-11mer (SEQ ID NO:4, SEQ ID NO: 2, SEQ ID NO: 16) in 50 mM NaCl, 10 mM MgCl₂, 0.1 M sodium phosphate pH 6.0). (See FIG. 23).

NMR Spectra

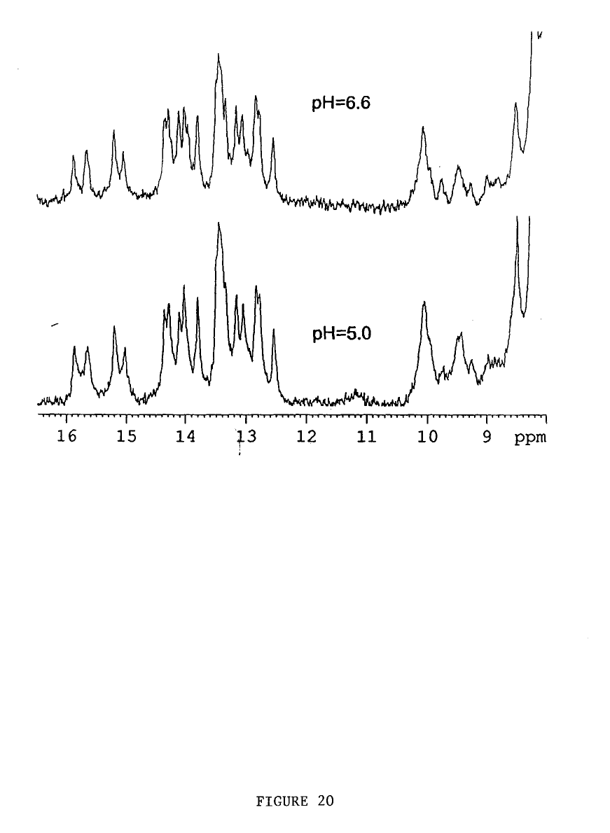

The imino region of one-dimensional ¹H-NMR spectra of the triplex formed by hairpin B-22A (SEQ ID NO:3, SEQ ID NO: 2) and polypyrimidine target WC-11mer (SEQ ID NO: 16) at two pHs is shown in FIG. 20. Most of the expected imino protons signals are clearly observed between 12 and 16 ppm. The presence of four imino signals between 15 and 16.0 ppm clearly indicates that cytosines are protonated. The spectrum is consistent with a triple helix. Most of the features of the exchangeable proton spectra are observed at about pH 6.6, which points to the high stability of the triplex at neutral pH. The lines of the exchangeable protons in this triplex are much narrower than in the isolated B-22A hairpin (SEQ ID NO:3, SEQ ID NO: 2). The line-broadening in the parallel Hoogsteen hairpin may be due to a conformational or solvent exchange. This dynamic effect is not observed upon triplex formation.

It will be understood by those persons skilled in the art that the present invention shows that the hairpins of this invention bind specific single-stranded polypyrimidine targets via triplex formation. The binding of these hairpins is stronger when they contain 8-aminopurines. 8-Aminopurine showed the strongest stabilizing effect, followed by 8-aminoadenine. 8-Aminohypoxanthine is more efficient than unmodified hairpin only at neutral pH. The stability of the triplex of this invention formed by hairpins carrying 8-aminopurines is pH-dependent but the interaction of the modified hairpins with their target is so strong that triplexes are observed even at neutral pH on a short model sequence such as for example having about 11 bases. Both 8-aminoadenine and 8-aminopurine have an additive effect on the stability of the triplex. The loop that connects the homopurine sequence with the homopyrimidine sequence may also have an additional stabilizing effect if it is made of nucleotides.

The modified hairpins may be redesigned to cope with small interruptions in the polypyrimidine target sequence. This offers great potential for applications in the triplex field, especially for single-stranded targets, e.g. in antisense field and RNA detection. The use of 8-aminopurines is also compatible with most of the developments described in the triplex field so we believe that 8-aminopurines will improve any existing methodology based on triplex formation.

TABLE 6
Melting temperatures* (C.) for the triplex formed by
R-22 derivatives and WC-11mer.^{SEQ ID NO: 16}

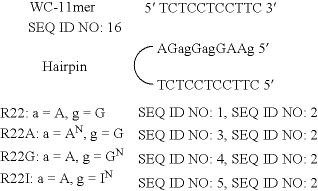

| Hairpin | Target | pH 4.5 | pH 5.5 | pH 6.0 | pH 6.5 | pH 7.0 |
|---|---|---|---|---|---|---|
| R-22*² | *⁶WC-11mer | 69 | 56 | 47 | 36 | 32 |
| R-22A*³ | *⁶WC-11mer | 73 | 62 | 56 | 48 | 45 |
| R-22G*⁴ | *⁶WC-11mer | 76 | 67 | 59 | 53 | 51 |
| R-22I*⁵ | *⁶WC-11mer | 65 | 34, 55 | 20, 46 | 40 | 38 |

*1 M NaCl, 100 mM sodium phosphate/citric acid buffer.
*²SEQ ID NO: 1, SEQ ID NO: 2
*³SEQ ID NO: 3, SEQ ID NO: 2
*⁴SEQ ID NO: 4, SEQ ID NO: 2
*⁵SEQ ID NO: 5, SEQ ID NO: 2
*⁶SEQ ID NO: 16

TABLE 7
Melting temperatures* (° C.) for the triplex formed by B-22 derivatives and
WC-11mer.^{SEQ ID NO: 16}

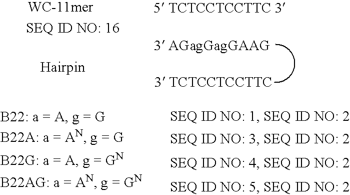

| Hairpin | Target | pH 4.6 | pH 5.5 | pH 6.0 | pH 6.5 | pH 7.0 |
|---|---|---|---|---|---|---|
| B-22*² | WC-11mer*⁶ | 63 | 54 | 45 | 33 | 20 |
| B-22A*³ | WC-11mer*⁶ | 73 | 57 | 51 | 43 | 34 |
| B-22G*⁴ | WC-11mer*⁶ | 75 | 69 | 59 | 50 | 40 |
| B-22AG*⁵ | WC-11mer*⁶ | 80 | 71 | 65 | 56 | 53 |

*1M NaCl, 100 mM sodium phosphate/citric acid buffer
*²SEQ ID NO: 1, SEQ ID NO: 2
*³SEQ ID NO: 3, SEQ ID NO: 2
*⁴SEQ ID NO: 4, SEQ ID NO: 2
*⁵SEQ ID NO: 5, SEQ ID NO: 2
*⁶SEQ ID NO: 16

TABLE 8
Effect of the Hoogsteen strand.

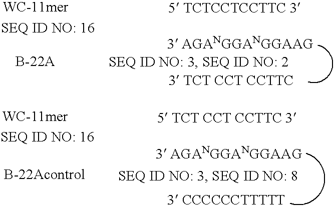

| | | pH 5.5, 1 M NaCl | | pH 6.0, 1 M NaCl | |
|---|---|---|---|---|---|
| Hairpin | Target | Tm (° C.) | Hyperchromicity | Tm (° C.) | Hyperchromicity |
| B-22Acontro*¹ | WC-11mer*⁵ | 41 | +12% | 40 | +11% |
| | | | (du to ss) | | (du to ss) |
| B-22Acontrol*² | none | No transition | | No transition | |
| B-22A*³ | WC-11mer*⁵ | 57 | +22% | 51 | +20% |
| | | | (tri to ss) | | (tri to ss) |
| B-22A*⁴ | none | 47 | +12% | 38 | +11% |
| | | | (du to ss) | | (du to ss) |

*¹SEQ ID NO: 3, SEQ ID NO: 8
*²SEQ ID NO: 3, SEQ ID NO: 8
*³SEQ ID NO: 3, SEQ ID NO: 2
*⁴SEQ ID NO: 3, SEQ ID NO: 2
*⁵SEQ ID NO: 16

TABLE 9
Melting temperatures of triplex containing one interruption at
the polypurine/polypyrimidine track (at pH 6.0, 0.1 M
sodium phosphate and citric acid, 1 M NaCl).

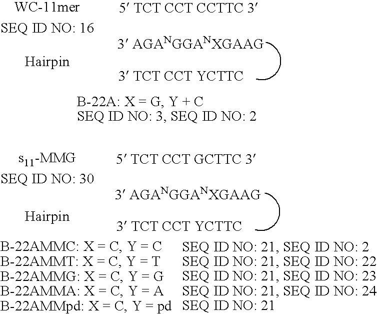

| | Target 1. WC-11mer | | Target 2. s₁₁-MMG | |
|---|---|---|---|---|
| | SEQ ID NO: 16 | | SEQ ID NO: 30 | |
| hairpin | Triad 1^{a} | Tm (° C.) | Triad 2^{a} | Tm (° C.) |
| B-22A*¹ | C.G-C | 51 | G.G-C | 43 |
| B-22AMMC*² | C.C-C | 33 | G.C-C | 47 |
| B-22AMMI*³ | C.C-T | 30 | G.C-T | 45 |
| B-22AMMG*⁴ | C.C-G | 34 | G.C-G | 43 |
| B-22AMMA*⁵ | C.C-A | 28 | G.C-A | 41 |
| B-22AMMpd*⁶ | C.C-pd | 29 | G.C-pd | 44 |

^{a}Watson-Crick base pair is indicated with a dot, Hoogsteen pair is indicated with a dash
*¹SEQ ID NO: 3, SEQ ID NO: 2
²SEQ ID NO: 21, SEQ ID No: 2
*³SEQ ID NO: 21, SEQ ID NO: 22
*⁴SEQ ID NO: 21, SEQ ID NO: 23
*⁵SEQ ID NO: 21, SEQ ID NO: 24
*⁶SEQ ID NO: 21

TABLE 10
Melting temperatures of triplex containing one interruption at
the polypurine/polypyrimidine track (at pH 6.0, 0.1 M sodium
phosphate and citric acid, 1 M NaCl).

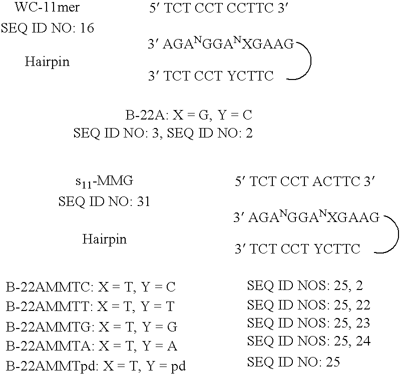

| | Target 1. WC-11mer | | Target 2. s₁₁-MMA | |
|---|---|---|---|---|
| | SEQ ID NO: 16 | | SEQ ID NO: 31 | |
| Hairpin | Triad 1^{a} | Tm (° C.) | Triad 2^{a} | Tm (° C.) |
| B-22A | C.G-C*¹ | 51 | G.G-C | 43 |
| B-22AMMTC | C.T-C*² | 28 | A.T-C | 39 |
| B-22AMMTT | C.T-T*³ | 31 | A.T-T | 40 |
| B-22AMMTG | C.T-G*⁴ | 33 | A.T-G | 46 |
| B-22AMMTA | C.T-A*⁵ | 31 | A.T-A | 40 |
| B-22AMMTp | C.T-pd*⁶ | 30 | A.T-pd | 42 |

^{a}Watson-Crick base pair is indicated with a dot, Hloogsteen pair is indicated with a dash
*¹SEQ ID NO: 3, SEQ ID NO: 2,
*²SEQ ID NO: 25, SEQ ID NO:2
*³SEQ ID NO: 25, SEQ ID NO: 22
*⁴SEQ ID NO: 25, SEQ ID NO: 23
*⁵SEQ ID NO: 25, SEQ ID NO: 24
*⁶SEQ ID NO: 25

TABLE 11
Folding processes and associated energies (in kcal/mol) computed in the
gas phase at the B3LYP/6-31G(d) level of theory. Watson-Crick base pair
is indicated with a dot, Hoogsteen base pair is indicated with a dash

| Folding process | Folding energy |
|---|---|
| A + T→A · T | −12.1 |
| A^{N} + T→A^{N} · T | −11.9 |
| G + C→GC | −25.1 |
| G^{N} + C→G^{N}C | −24.8 |
| (T − A) + T→T − A · T | −10.8 |
| (T − A^{N}) + T→T − A^{N} · T | −11.6 |
| (C⁺ −G) + C→C⁺ − GC | −28.1 |
| (C⁺ − G^{N}) + C→C⁺ − G^{N}C | −27.0 |

TABLE 12
Thermodynamic parameters for triplex to random coil transitions in
sodium acetate 100 mM (pH 6.0)), 50 mM NaCl, 10 mM MgCl₂ from the
slope of the plot 1/‘1’m versus In C^{a}).

| | Tm | | | ΔGₜ |
|---|---|---|---|---|
| triplex | (° C.)^{b)} | ΔHₜ (kJ/mol) | ΔSₜ (J/mol K) | (kJ/mol) |
| B-22 + WC-11mer*¹ | 34.5 | −731 | −2258 | −58 |
| B-22A + WC-11mer*² | 52.5 | −498 | −1416 | −76 |
| B-22G + WC-11mer*³ | 57.3 | −554 | −1562 | −88 |

^{a)}ΔHₜ, ΔSₜ and ΔGₜ are given as round number, ΔGₜ is calculated at 25° C., with the assumption that ΔHₜ and ΔSₜ do not depend on temperature; analysis has been carried out using melting temperatures obtained from denaturation curves; error on Tm is 0.7° C.
^{b) at 4 μM triplex concentration}
*¹SEQ ID NO: 1, SEQ ID NO: 2, SEQ ID NO: 16
*²SEQ ID NO: 3, SEQ ID NO: 2, SEQ ID NO: 16
*³SEQ ID NO: 4, SEQ ID NO: 2, SEQ ID NO: 16

Whereas, particular embodiments of this invention have been described for purposes of illustration, it will be evident to those persons skilled in the art that numerous variations of the details of the present invention may be made without departing from the invention as defined in the appended claims and SEQUENCE LISTING.

We claim:

1. A triplex comprising:
a hairpin comprising at least one first polypyrimidine sequence, at least one linker, and at least one polypurine sequence, wherein at least one of said polypurine sequence is complementary to and parallel to said first polypyrimidine sequence, and said polypurine sequence comprising at least one 8-aminopurine;
at least one polypyrimidine target sequence, wherein at least one of said polypyrimidine target sequence is complementary to and antiparallel to said polypurine sequence, wherein said potypyrimidine target sequence and said hairpin are bound to each other.

2. The triplex of claim 1 wherein said polypyrimidine target sequence comprises at least one purine interruption.

3. The triplex of claim 1 wherein said polypurine sequence of said hairpin comprises at least one pyrimidine interruption.

4. The triplex of claim I wherein said first polypyrimidine sequence of said hairpin comprises at least one purine interruption or an abasic interruption or an abasic model compound interruption.

5. The triplex of claim 1 wherein said linker is at least one of a hexaethylene glycol, tetrathymine, CTTTG, or GGAGG.

6. The triplex of claim 1 wherein said 8-aminopurine comprises 8-aminopurine.

7. The triplex of claim 1 wherein said 8-aminopurine comprises 8-aminoadenine.

8. The triplex of claim 1 wherein said 8-aminopurine comprises 8-aminohypoxanthine.

9. A method for preparing a hairpin containing at least one 8-aminopurine comprising:
preparing a pyrimidine strand;
binding a linker to the 3′ end of said pyrimidine strand;
preparing a purine strand comprising at least one 8-aminopurine; and
preparing said hairpin by binding the 3′ end of said purine strand to said linker.

10. A method for preparing a hairpin containing at least one 8-aminopurine comprising:
preparing a purine strand comprising at least one 8-aminopurine;
binding a linker to the 5′ end of said purine strand;
preparing a pyrimidine strand; and
preparing said hairpin by binding the 5′ end of said pyrimidine strand to said linker.

11. A hairpin comprising at least one first polypyrimidine sequence, at least one linker, and at least one polypurine sequence, wherein at least one of said polypurine sequence comprises at least one 8-aminopurine and wherein said polypurine sequence is complementary to and parallel to said first polypyrimidine sequence.

12. A method for stabilizing a triplex comprising:
obtaining a triplex comprising a hairpin comprising at least a first polypyrimidine sequence, at least one linker, and at least one polypurine sequence, wherein said polypurine sequence comprises at least one 8-aminopurine; and
contacting said triplex with a sodium chloride solution.

13. The method of Claim 12 wherein said sodium chloride solution has a concentration of about 1 M.

14. A method for stabilizing a triplex comprising:
obtaining a triplex comprising a hairpin comprising at least a first polypyrimidine sequence, at least one linker, and at least one polypurine sequence wherein said polypurine sequence comprises at least one 8-aminopurine; and
contacting said triplex with a magnesium containing solution.

15. The method of claim 14 including wherein the concentration of said magnesium is not greater than about 10 mM.

16. A triplex comprising:
a hairpin comprising at least one first polypyrimidine sequence, at least one linker, and at least one first polypurine sequence wherein said polypurine sequence is complementary to and antiparallel to said first polypyrimidine sequence, and said first polypurine sequence comprising at least one 8-aminopurine; and
a target sequence wherein said target sequence is arranged in Hoogsteen orientation with respect to said hairpin.

17. The triplex of claim 16 wherein said target sequence comprises G and T bases.

18. The triplex of claim 16, wherein said target sequence comprises G and A bases.

19. An oligonucleotide duplex comprising two complementary oligonucleotide strands arranged in an anti-parallel Hoogsteen configuration.

20. A method for stabilizing Hoogsteen duplexes comprising:
procuring a Hoogsteen duplex comprising at least one purine; and
stabilizing said Hoogsteen duplex by substituting at least one 8-aminopurine for at least one of said purine.

21. A method for targeting a single-stranded oligonucleotide comprising:
selecting a region on a single-stranded oligonucleotide, said region having either a first polypurine sequence target or a first polypyrimidine sequence target;
preparing a hairpin wherein said hairpin comprises a second polypyrimidine sequence and a second polypurine sequence, wherein said second polypurine sequence comprises at least one 8-aminopurine and is complementary to said second polypyrimidine sequence; and
targeting said region on said single-stranded oligonucleotide by contacting said hairpin with said first polypurine sequence target or said first polypyrimidine sequence target.

22. The method of claim 21, wherein said single-stranded oligonucleotide is selected from the group consisting of cDNA, MRNA, tRNA, and rRNA.

23. A method for targeting DNA comprising:
selecting a region on DNA, said region having either a first polypurine sequence target or a first polypyrimidine sequence target;
preparing a hairpin wherein said hairpin comprises a second polypyrimidine sequence and a second polypurine sequence, wherein said second polypurine sequence comprises at least one 8-aminopurine and is complementary to said second polypyrimidine science; and
targeting the region on said DNA by contacting said hairpin with said first polypurine sequence target or said first polypyrimidine sequence target.
